(19) United States
(12) Patent Application Publication
(10) Pub. No.: US 2009/0208938 A1
(43) Pub. Date: Aug. 20, 2009

(54) Use of the MCM8 Gene for the Preparation of a Pharmaceutical Composition

(75) Inventors: Marchel Mechali, Montferrier Sur Lez (FR); Domenico Maiorano, Saint -Martin De Londres (FR)

Correspondence Address: YOUNG & THOMPSON, 209 Madison Street, Suite 500, ALEXANDRIA, VA, 22314, US

(73) Assignee: CENTRE NATIONAL DE LA RECHERCHE SCIENTIFIQUE, PARIS (FR)

(21) Appl. No.: 11/884,053

(22) PCT Filed: Feb. 9, 2006
(86) PCT No.: PCT/EP2006/001158

§ 371 Date: Sep. 17, 2008

Related U.S. Application Data
(60) Provisional application No. 60/651,510, filed on Feb. 10, 2005.

(51) Int. Cl. C12Q 1/68; A61K 31/7105; A61K 38/43; A61K 39/395; C12N 5/06; C12P 21/02; C12N 15/63; C12N 5/10; C12N 9/00; C07K 16/40

(52) U.S. Cl. 435/6; 424/94.1; 424/158.1; 435/375; 435/69.1; 435/68.1; 435/320.1; 435/325; 435/183; 530/387.9; 530/387.3; 514/44 A

(57) ABSTRACT

The use of the human or animal MCM8 gene coding for a DNA helicase, or parts of the gene, or transcripts thereof, or antisense nucleic acids able to hybridize with part of the transcripts, or silencing RNA derived from parts of the transcripts and able to repress the MCM8 gene, or proteins or peptidic fragments translated from the transcripts, or antibodies directed against the proteins or peptidic fragments for the preparation of a pharmaceutical composition for the treatment of a human or animal pathology linked to a dysfunction of the expression of the MCM8 gene, or of human or animal cancers.

The present invention relates to the use of the MCM8 gene, in particular in the pharmaceutical field.

The duplication of the eukaryotic genome is achieved through the assembly of efficient replication machineries. This process is initiated by the Origin Recognition Complex (ORC) binding to DNA replication origins. Pre-replication (pre-RCs) and pre-initiation (pre-ICs) complexes are then formed, during a series of sequential reactions leading to assembly of replication forks (Bell and Dutta, 2002) for review). Assembly of pre-RCs depends upon the Cdc6 and Cdt1 proteins, resulting in recruitment of MCM2-7 proteins at DNA replication origins (the licensing reaction). Geminin (McGarry and Kirschner, 1998) blocks pre-RC formation by interfering with the activity of Cdt1 (Tada et al., 2001; Wohlschlegel et al., 2000). Three additional factors, the Cdc7 protein kinase, Cut5 and the MCM10 proteins (this latter being unrelated to the MCM2-7 protein family) are then recruited (Mendez and Stillman, 2003) for review). Formation of pre-ICs requires previous assembly of pre-RCs and S-CDK activity, and is catalyzed by the Cdc45 protein, in combination with the GINS complex (Mendez and Stillman, 2003). This reaction is specifically inhibited by the CDK inhibitor p21. Cdc45 allows assembly of initiation complexes by recruitment of DNA polymerases at replication origins (Mimura et al., 2000; Mimura and Takisawa, 1998; Walter and Newport, 2000).

Anomalies during DNA replication process are involved in different pathologies such as brains diseases, haematological disorders and cancers. Thus, means to control cellular division would be useful tools for the treatment of pathologies linked to a dysfunction of DNA replication or for pathologies linked to an excessive cellular proliferation.

Components of the replication fork include the trimeric, single-stranded DNA binding RPA complex, and the DNA helicase. These latter would represent ideal targets to achieve a control of the DNA replication process but the identity of the DNA helicases that function at replication forks remains debated. Genetic and biochemical evidence support a role for the MCM2-7 protein family providing helicase activity in unwinding DNA at replication origins during initiation (Kearsey and Labib, 1998; Labib and Diffley, 2001; Tye, 1999) for review). The MCM2-7 proteins form a stable complex in vitro, although detectable helicase activity is only observed with the MCM4, 6, 7 sub-complex (Ishimi, 1997). Current models suggest that this sub-complex may represent the active helicase, while the remaining subunits may have an essential role in regulating the activity of the helicase (Davey et al., 2003; Ishimi et al., 1998; Schwacha and Bell, 2001). The functional association of MCM2-7 with chromatin is cell cycle-regulated. These proteins are synchronously recruited to both early and late DNA replication origins immediately after mitotic exit (Dimitrova et al., 1999) and are removed from chromatin in S phase (Kearsey and Labib, 1998) for review).

A role for MCM2-7 has also been suggested during the elongation step. In budding yeast, MCM4 appears to move away from replication origins after initiation of DNA synthesis (Aparicio et al., 1997; Tanaka et al., 1997). Moreover, genetic data indicate that all MCM2-7 are required for replication throughout S-phase (Labib et al., 2000). However a number of observations contrast with this conclusion. First, MCM2-7 bind preferentially unreplicated DNA and are gradually displaced from chromatin during replication fork movement (Kubota et al., 1995; Labib et al., 1999; Madine et al., 1995b; Todorov et al., 1995). Second, interactions between the replicative helicase and components of the replication fork are predicted by experiments carried out with the Simian Virus-40 eukaryotic in vitro system for DNA replication (Dorneiter, 1992; Melendy and Stillman, 1993; Waga, 1994). However no physical interaction between MCM2-7 and components of the DNA synthesis machinery has been observed, such as interactions with the RPA complex and DNA polymerases. Finally, MCM2-7 do no co-localize with DNA replication foci (Coué et al., 1996; Dimitrova et al., 1999; Krude et al., 1996; Madine et al., 1995b; Romanowski et al., 1996). To explain this paradox it was proposed that the helicase activity of MCM2-7 proteins may only be required at the initial step of DNA unwinding, and that another helicase may take over the role of MCM2-7 during elongation (Ishimi, 1997). More recently, a model has been proposed (Laskey and Madine, 2003), in which MCM2-7 proteins may. act as rotary pumps in unwinding (Schwacha and Bell, 2001) at a fixed position, away from replication forks.

An additional member of the MCM2-7 family, HMCM8, has been described in human cells (Gozuacik et al., 2001). HMCM8 is stable throughout the cell cycle (Gozuacik et al., 2003), binds to chromatin later than HMCM3 and does not associate with HMCM2-7 proteins in vitro. However, an independent study has reported that a fraction of HMCM8 might associate with MCM4, 6, 7 proteins in Hela cells (Johnson et al., 2003). These observations have suggested a role of MCM8 in S-phase, but its function remains unknown.

The present invention relates to the use of the MCM8 gene in pathologies linked to a dysfunction of DNA replication or to an excessive cell proliferation.

The invention also provides a method for inhibiting cell proliferation or enhancing DNA replication.

The invention provides a method for screening drugs useful in the treatment of pathologies linked to a dysfunction of the replication or to an excessive cell proliferation.

Another aspect of the invention relates to pharmaceutical compositions comprising a MCM8 protein or a polypeptide comprising part of said protein.

The present invention relates to the use of the human or animal MCM8 gene coding for a DNA helicase, or parts of said gene, or transcripts thereof, or antisense nucleic acids able to hybridize with part of said transcripts, or silencing RNA derived from parts of said transcripts and able to repress said MCM8 gene, or proteins or peptidic fragments translated from said transcripts, or antibodies directed against said proteins or peptidic fragments for the preparation of a pharmaceutical composition for the treatment of a human or animal pathology linked to a dysfunction of the expression of the MCM8 gene, or of human or animal cancers.

The inventors have described the identification and biochemical characterization of MCM8, an MCM2-7-related protein, which is widely conserved in vertebrates (Gozuacik et al., 2003). MCM8 functions as a DNA helicase at replication forks during the elongation step of DNA synthesis and may have a similar role in other vertebrates.

DNA helicases have essential roles in nucleic acid metabolism, particularly during DNA replication, also called DNA duplication. Helicases are involved in unwinding DNA at replication origins, allowing DNA synthesis by recruiting DNA polymerases and they are also involved in the whole process of the elongation and termination phases of DNA synthesis when DNA has to be continuously and efficiently unwound. DNA helicases bind to single strand DNA either naked or coated with the single strand DNA binding protein RPA as oligomeric complexes and catalyze the melting of the DNA double helix. This reaction is catalyzed by ATP hydrolysis.

The helicase activity of a protein can be for example determined by the following test: the protein to test is incubated with a single-stranded DNA substrate annealed to a 40-mer oligonucleotide for 1 hour. The reaction products are then separated on an acrylamide gel. The helicase activity is revealed by the presence of single strand DNA, due to the unwinding of the dimer single-stranded DNA/oligonucleotide.

The expression “dysfunction of the expression of the MCM8 gene” relates to an overexpression, a repression or an inhibition of the expression of the MCM8 gene, or relates to the expression of a protein coded by the MCM8 gene, which is not active or only partially active. A dysfunction of the MCM8 gene expression can particularly induce disorders in DNA replication.

The dysfunction of the expression of the MCM8 gene can be assayed by the determination of the amount of MCM8 mRNA produced in the cell either by hybridization of total cellular RNA with either a DNA or RNA probe derived from the sequence of the MCM8 gene (Northern blot) or by PCR amplification of the MCM8 mRNA, following its conversion into cDNA by the use of a Reverse Transcriptase (RT-PCR), or by in situ hybridization with either DNA or RNA probes derived from the sequence of the MCM8 gene after fluorescent labelling of these probes. MCM8-specific antibodies can be also used to determine the levels of the MCM8 protein present in cells and/or tissues by western or by in situ hybridization on fixed tissues slices of isolated cells and/or nuclei.

The expression “pathologies linked to a dysfunction of the expression of the MCM8 gene” means that these pathologies result from disorders in helicase activity of the MCM8 gene.

The expression “parts of said gene” means fragments of the MCM8 gene.

The invention also relates to the use of transcripts of the MCM8 gene or of parts of the MCM8 gene. The translation of these transcripts, also called mRNAs, will produce the MCM8 protein, or peptidic fragments of said protein. The proteins or peptidic fragments can be purified from cells expressing said compounds. The peptidic fragments according to the invention can also be synthesized by any method of chemistry well-known in the art.

The invention further relates to the use of antisense nucleic acids. Antisense nucleic acids, also called antisense-oligonucleotides (AS-ONs) pair with their complementary mRNA target, thus blocking the translation of said MRNA or inducing the cleavage by RNase H of said mRNA inside the DNA/RNA complex. In both cases, the use of antisense nucleic acids induces a specific blocking of RNA translation. The antisense nucleic acids according to the invention comprise preferentially 10 to 30 nucleotides. The use of antisense nucleic acids able to hybridize with transcripts of the MCM8 gene thus allows inhibiting the expression of the MCM8 gene.

The invention also relates to the use of silencing RNA, also called interfering RNA, derived from parts of transcripts of the MCM8 gene. RNA interference is a process initiated by double-strand RNA molecules (dsRNAs), which are cut by the cell machinery into 21-23 nucleotides long RNAs, called small interfering RNAs (siRNAs). In the cell, said siRNAs are then incorporated into RNA-Induced Silencing Complex (RISC), in which they guide a nuclease to degrade the target simple strand RNA. The use of silencing RNAs, which are complementary to parts of MCM8 transcripts, allows the specific inhibition of the MCM8 expression.

The invention also relates to the use of antibodies directed against MCM8 proteins or peptidic fragments of said protein. These antibodies thus bind to the MCM8 protein in the cell, thus inhibiting its helicase function.

The invention relates in particular to the use as defined above for the preparation of a pharmaceutical composition for the treatment of cancers, wherein the helicase activity of MCM8 in tumoral cells of the human or animal body is inactivated by using silencing iRNA according to RNA interference, such as double-stranded RNA (dsRNA) for post-transcriptional gene silencing, or short interfering RNA (siRNA) or short hairpin RNA (shRNA) to induce specific gene suppression, or antisense DNA or RNA, or antibodies, in order to curb the proliferation of said tumoral cells.

In a particular embodiment, the invention aims at inhibiting the proliferation of cancer cells. For that purpose, the helicase activity in tumoral cells is inactivated by specifically blocking MCM8 expression using RNA interference or antisense nucleotides, or by blocking the MCM8 protein with specific antibodies. The level of active MCM8 and consequently the level of helicase activity are decreased and the DNA replication is curbed. The proliferation of the tumoral cells is thus inhibited and a stop of the DNA replication process may also induce apoptosis of the tumoral cells.

The efficiency of inhibition of the helicase activity can be determined by cell proliferation test. For example, classical tests based on BrdU incorporation during DNA synthesis can be used or other tests such as analysis of the DNA content of a cell population by Fluorescence Activated Cell Sorter (FACS), or by incorporation of either a radioactively labelled DNA precursor, or H³ (tritium) into thrichloroacetic acid (TCA) insoluble material, or by scoring the mitotic index of a cell population, or by scoring the increase in the total mass of a cell population (growth curve), or the increase in the rate of protein synthesis, or by scoring the number of Ki67-, PCNA-, MCM2-7- or Cdc6-positive cells.

For the purpose of the invention, the RNA interference is obtained by using interfering RNA chosen among double-strand RNA, short interfering RNA or short hairpin RNA. Interfering RNA can be obtained by chemical synthesis or by DNA-vector technology.

A short hairpin RNA is a simple strand RNA, characterized in that the two ends of said RNA are complementary and can hybridize together, thus forming an artificial double strand RNA with a loop between the two ends.

The invention further relates to the use as defined above for the preparation of a pharmaceutical composition for the treatment of neoplastic diseases such as choriocarcinoma, liver cancer induced by DNA damaging agents or by infection by Hepatitis B virus, skin melanotic melanoma, melanoma, premalignant actinic keratose, colon adenocarcinoma, basal cell carcinoma, squamous cell carcinoma, ocular cancer, non-Hodgkin's lymphoma, acute lymphocytic leukaemia, meningioma, soft tissue sarcoma, osteosarcoma, and muscle rhabdomyosarcoma or of brain diseases such as Alzheimer disease, neuron degenerative diseases and mental retardation, or of haematological disorders.

The invention also relates to the above-mentioned use for the preparation of a pharmaceutical composition for the treatment of a human or animal pathology linked to a dysfunction of the expression of the MCM8 gene, wherein the number of functional MCM8 helicases is increased or the activity of MCM8 helicases in cells of the human or animal body is stimulated by administration of functional MCM8 proteins or of fragments thereof or by gene or cell therapy.

The above-mentioned pathologies result from the absence or the small rate of helicase activity of the MCM8 protein, which may result from the expression of an inactive form of the MCM8 protein or from an expression of said protein which is between 1% to 60% smaller than the expression in normal cell.

The increased number of functional helicases can be determined by immunoblot with MCM8 specific antibodies on total cell lysates, or by in situ immunostaining on a given cell population or a tissue and/or by isolation of the MCM8 protein by immunopurification with MCM8-specific antibodies and determination of both helicase and ATPase activity in vitro compared to normal cells.

The stimulation of the MCM8 helicase activity is determined by performing an helicase test as described above in the presence of the single strand DNA annealed to an oligonucleotide, the single strand DNA binding trimeric complex RPA, or with DNA polymerases, PCNA, RF-C and/or other replication fork accessory proteins.

The expression “trimeric complex” means a protein complex made of three polypeptides.

The term “gene therapy” refers to the use of DNA as a drug. According to the invention, said DNA comprises the MCM8 gene and is introduced in the cells so that they can express the MCM8 protein. Gene transfer methods are well-known by the man skilled in the art. They comprise physical methods, such as naked DNA, microinjection, shotgun or electrotransfer, and vectorization using non-viral or viral vectors for the gene transfer.
the human MCM8 nucleotide sequence represented by SEQ ID NO: 7 encoding the human helicase represented by SEQ ID NO:8,
the human MCM8 nucleotide sequence represented by SEQ ID NO: 9 encoding the human helicase represented by SEQ ID NO: 10,
the human MCM8 nucleotide sequence represented by SEQ ID NO: 11 encoding the human helicase represented by SEQ ID NO: 12,
the human MCM8 nucleotide sequence represented by SEQ ID NO: 13 encoding the human helicase represented by SEQ ID NO: 14,
the human MCM8 nucleotide sequence represented by SEQ ID NO: 15 encoding the human helicase represented by SEQ ID NO: 16,
the murine MCM8 nucleotide sequence represented by SEQ ID NO: 17 encoding the murine helicase represented by SEQ ID NO: 18,
the murine MCM8 nucleotide sequence represented by SEQ ID NO:19 encoding the murine helicase represented by SEQ ID NO: 20,
the murine MCM8 nucleotide sequence represented by SEQ ID NO: 21 encoding the murine helicase represented by SEQ ID NO: 22.

The present invention also relates to nucleotide sequences which encode the above described proteins due to the degeneracy of the genetic code.

The invention also relates to homologous nucleotide sequences, which have at least 75% of identity with the above described nucleotide sequences, particularly at least 90% and more particularly at least 95% of identity, and which encode proteins that have a helicase activity, and also relates to said proteins.

SEQ ID NO: 1 and 2 correspond to the Xenopus MCM8 gene and protein sequence, respectively (accession number AJ867218).

SEQ ID NO: 3 and 4 correspond to the human MCM8 gene and protein sequence, respectively (accession number BC005170).

SEQ ID NO: 5 and 6 correspond to the human MCM8 gene and protein sequence, respectively (accession number NM_{—}182802).

SEQ ID NO: 7 and 8 correspond to the human MCM8 gene and protein sequence, respectively (accession number NM_{—}032485).

SEQ ID NO: 9 and 10 correspond to the human MCM8 gene and protein sequence, respectively (accession number BC080656).

SEQ ID NO: 11 and 12 correspond to the human MCM8 gene and protein sequence, respectively (accession number BC008830).

SEQ ID NO: 13 and 14 correspond to the human MCM8 gene and protein sequence, respectively (accession number AY158211).

SEQ ID NO: 15 and 16 correspond to the human MCM8 gene and protein sequence, respectively (accession number AJ439063).

SEQ ID NO: 17 and 18 correspond to the murine MCM8 and protein sequence, respectively (accession number BC046780).

SEQ ID NO: 19 and 20 correspond to the murine MCMS gene and protein sequence, respectively (accession number BC052070).

SEQ ID NO: 21 and 22 correspond to the murine MCM8 gene and protein sequence, respectively (accession number NM_{—}025676).

Human nucleotide sequences SEQ ID NO: 9, 15 correspond to the wild type HMCM8 sequences and the human protein sequences SEQ ID NO: 10 and 16 have 840 amino-acids.

SEQ ID NO: 3 (BC005170) and SEQ ID NO: 5 (NM_{—}182802) differ from the wild-type HMCM8 sequence in a deletion of 47 base pairs in the MCM8 cDNA, resulting in a deletion of 16 amino acids in the corresponding protein (from amino acids 331 to 348 of the wild-type MCM8 protein). The corresponding human protein sequences SEQ ID NO: 4 and 6 have 824 amino-acids.

SEQ ID NO: 7 (NM_{—}032485) differs in the length of the 3′ untranslated region of the MCM8 cDNA.

SEQ ID NO: 13 (AY158211) is an isoform produced by aberrant splicing in exon 10 in choriocarcinoma cells, resulting in a deletion of 47 base pairs in the MCM8 cDNA and resulting in a deletion of 16 amino acids in the corresponding protein (from amino acids 331 to 348 of the wild-type MCM8 protein).

Human protein sequence SEQ ID NO:12 corresponds to a truncated form of the 840 amino-acid long protein, wherein the first 105 amino-acids are missing.

Murine protein sequences SEQ ID NO: 20 and 22 have 805 amino-acids and murine protein sequence SEQ. ID NO: 18 has 833 amino-acids. SEQ ID NO: 20 and 22 differ from SEQ ID NO 18 by a deletion of 28 amino acids and by 12 polymorphic amino acids.

The present invention further relates to the use as defined above, wherein said parts of the MCM8 nucleotide sequence contain approximately 3 to 240 nucleotides, and comprise a segment which is essential for the helicase function of MCM8 protein, said segment being notably selected from the group composed of:
the nucleotide sequence represented by SEQ ID NO: 23, corresponding to nucleotides 1345-1368 of the xenopus MCM8 gene represented by SEQ BD NO: 1,
the nucleotide sequence represented by SEQ ID NO: 25, corresponding to nucleotides 1537-1548 of the xenopus MCM8 gene represented by SEQ ID NO: 1,
the nucleotide sequence represented by SEQ ID NO: 27, corresponding to nucleotides 1360-1383 of the human MCM8 gene represented by SEQ ID NO: 9 or SEQ ID: 15,
the nucleotide sequence represented by SEQ ID NO: 29, corresponding to nucleotides 1552-1563 of the human MCM8 gene represented by SEQ ID NO: SEQ ID NO: 9 or SEQ ID: 15,
the nucleotide sequence represented by SEQ ID NO: 31, corresponding to nucleotides 1312-1338 of the human MCM8 gene represented by SEQ ID NO: 3 or SEQ ID NO:13,
the nucleotide sequence represented by SEQ ID NO: 33, corresponding to nucleotides 1504-1515 of the human MCM8 gene represented by SEQ ID NO: 3 or SEQ ID NO: 13,
the nucleotide sequence represented by SEQ ID NO: 35, corresponding to nucleotides 1339-1362 of the murine MCM8 gene represented by SEQ ID NO: 17,
the nucleotide sequence represented by SEQ ID NO: 37, corresponding to nucleotides 1531-1542 of the murine MCM8 gene, represented by SEQ ID NO:17,
the nucleotide sequence represented by SEQ ID NO: 39, corresponding to nucleotides 1255-1278 of the murine MCM8 gene, represented by SEQ ID NO: 19,
the nucleotide sequence represented by SEQ ID NO: 41, corresponding to nucleotides 1447-1458 of the murine MCMS gene, represented by SEQ ID NO: 19,

or wherein said peptidic fragments contain approximately 4 to 90 amino acids, and comprise a segment which is essential for the helicase function of MCM8 protein and which is notably selected from the group composed of:
the amino-acid sequence represented by SEQ ID NO: 24, corresponding to amino acids 449-456 of the xenopus MCM8 protein represented by SEQ ID NO: 2,
the amino-acid sequence represented by SEQ ID NO: 26, corresponding to amino acids 513-516 of the xenopus MCM8 protein represented by SEQ ID NO: 2,
the amino-acid sequence represented by SEQ ID NO: 28, corresponding to amino acids 454-461 of the human MCM8 protein represented by SEQ ID NO: 10 or SEQ ID: 16,
the amino-acid sequence represented by SEQ ID NO: 30, corresponding to amino acids 518-521 of the human MCM8 protein represented by SEQ ID NO: 10 or SEQ ID: 16,
the amino-acid sequence represented by SEQ ID NO: 32, corresponding to amino acids 438-446 of the human MCM8 protein represented by SEQ ID NO: 4 or SEQ ID: 14,
the amino-acid sequence represented by SEQ ID NO: 34, corresponding to amino acids 502-505 of the human MCM8 protein represented by SEQ ID NO: 4 or SEQ ID: 14,
the amino-acid sequence represented by SEQ ID NO: 36, corresponding to amino acids 447-454 of the murine MCM8 protein represented by SEQ ID NO: 18,
the amino-acid sequence represented by SEQ ID NO: 38, corresponding to amino acids 511-514 of the murine MCM8 protein represented by SEQ ID NO: 18,
the amino-acid sequence represented by SEQ ID NO: 40, corresponding to amino acids 419-426 of the murine MCM8 protein represented by SEQ ID NO: 20,
the amino-acid sequence represented by SEQ ID NO: 42, corresponding to amino acids 483-486 of the murine MCM8 protein represented by SEQ ID NO: 20.

The term “parts of the MCM8 nucleotide sequence” refers to fragments of the MCM8 gene that contain approximately 3 to 240 contiguous nucleotides.

The expression “segment which is essential for the helicase function of MCM8 protein” refers particularly to the Walker A motif and the Walker B motif.

Walker A motif is involved in ATP binding. This motif forms a Glycin-rich flexible loop preceded by a β-strand and followed by an α-helix. The Walker A motif of Xenopus and mammalian MCM8 homologs (Gozuacik et al., 2003; Johnson et al., 2003) is a canonical consensus sequence (GxxGxGKS/T).

Walker B motif is involved in ATP hydrolysis and has the following structure: hybrophobic stretch followed by the amino acids signature D[ED], where the presence of at least one negatively charged amino acid in this motif is crucial for its function.

According to another embodiment, the present invention relates to the use as defined above, wherein said MCM8 gene or said parts of the MCM8 nucleotide sequence or said transcripts or said proteins or peptidic fragments contain at least one mutation, by deletion and/or addition and/or substitution of one or more nucleotide or amino-acid.

The mutation by deletion or by addition in the nucleic acid can eventually induce a shift in the opening reading frame of the MCM8 nucleotide sequence.

The mutation by substitution in the protein or peptidic fragment or the mutation can be a substitution by a conservative amino-acid or not.

The mutation by substitution in the nucleotide sequence can lead to a silencing substitution due to the degeneracy of the genetic code, or to a substitution by a conservative amino-acid or a non conservative amino-acid in the protein or peptidic fragment encoded by said nucleotide sequence.

The present invention also relates to the use as defined above, wherein said mutation is located on a site of phosphorylation by CDKs, said site being notably selected from the group composed of:
nucleotides 253-258 of the xenopus MCM8 gene represented by SEQ ID NO: 1, encoding amino-acids 85-86 of the xenopus MCM8 protein represented by SEQ ID NO: 2,
nucleotides 820-825 of the xenopus MCM8 gene represented by SEQ ID NO: 1, encoding amino-acids 274-275 of the xenopus MCM8 protein represented by SEQ ID NO: 2,
nucleotides 1771-1776 of the xenopus MCM8 gene represented by SEQ ID NO: 1, encoding amino-acids 591-592 of the xenopus MCM8 protein represented by SEQ ID NO: 2,
nucleotides 2026-2031 of the xenopus MCM8 gene represented by SEQ ID NO: 1, encoding amino-acids 676-677 of the xenopus MCM8 protein represented by SEQ ID NO: 2,
nucleotides 2098-2103 of the xenopus MCM8 gene represented by SEQ ID NO: 1, encoding amino-acids 700-701 of the xenopus MCM8 protein represented by SEQ ID NO: 2,
nucleotides 154-159 of the human MCM8 gene represented by SEQ ID NO: 9 or SEQ ID NO: 15, encoding amino-acids 52-53 of the human MCM8 protein represented by SEQ ID NO: 10 or SEQ ID NO: 16,
nucleotides 181-186 of the human MCM8 gene represented by SEQ ID NO: 9 or SEQ ID NO: 15, encoding amino-acids 61-62 of the human MCM8 protein represented by SEQ ID NO: 10 or SEQ ID NO: 16,
nucleotides 268-273 of the human MCM8 gene represented by SEQ ID NO: 9 or SEQ ID NO: 15, encoding amino-acids 90-91 of the human MCM8 protein represented by SEQ ID NO: 10 or SEQ ID NO: 16,
nucleotides 838-843 of the human MCM8 gene represented by SEQ ID NO: 9 or SEQ ID NO: 15, encoding amino-acids 280-281 of the human MCM8 protein represented SEQ ID NO: 10 or SEQ ID NO: 16,
nucleotides 1786-1791 of the human MCM8 gene represented by SEQ ID NO: 9 or SEQ ID NO: 15, encoding amino-acids 596-597 of the human MCM8 protein represented SEQ ID NO: 10 or SEQ ID NO: 16,
nucleotides 2116-2121 of the human MCM8 gene represented by SEQ ID NO: 9 or SEQ ID NO: 15, encoding amino-acids 706-707 of the human MCM8 protein represented by SEQ ID NO: 10 or SEQ ID NO: 16,
nucleotides 1738-1743 of the human MCM8 gene represented by SEQ ID NO: 3 or SEQ ID NO: 13, encoding amino-acids 580-581 of the human MCM8 protein represented by SEQ ID NO: 4 or SEQ ID NO: 14,
nucleotides 2068-2073 of the human MCM8 gene represented by SEQ ID NO: 3 or SEQ ID NO: 13, encoding amino-acids 690-691 of the human MCM8 protein represented by SEQ ID NO: 4 or SEQ ID NO: 14,
nucleotides 247-252 of the murine MCM8 gene represented by SEQ ID NO: 17, encoding amino-acids 83-84 of the murine MCM8 protein represented by SEQ ID NO: 18,
nucleotides 817-822 of the murine MCM8 gene represented by SEQ ID NO: 17, encoding amino-acids 273-274 of the murine MCM8 protein represented by SEQ ID NO: 18,
nucleotides 1765-1770 of the murine MCM8 gene represented by SEQ D) NO: 17, encoding amino-acids 589-590 of the murine MCM8 protein represented by SEQ ID NO: 18,
nucleotides 163-168 of the murine MCM8 gene represented by SEQ ID NO: 19, encoding amino-acids 55-56 of the murine MCM8 protein represented by SEQ ID NO: 20,
nucleotides 733-738 of the murine MCM8 gene represented by SEQ ID NO: 19, encoding amino-acids 245-246 of the murine MCM8 protein represented by SEQ ID NO: 20,
nucleotides 1681-1686 of the murine MCM8 gene represented by SEQ ID NO: 19, encoding amino-acids 561-562 of the murine MCM8 protein represented by SEQ ID NO: 20,
and nucleotides 2011-2016 of the murine MCM8 gene represented by SEQ ID NO: 19, encoding amino-acids 671-672 of the murine MCM8 protein represented by SEQ ID NO: 20.

CDKs (Cyclin-Dependent Kinases) are enzymes involved in the regulation of cell division cycle. CDKs activate their substrate by phosphorylation. CDKs recognize specific sites, called “site of phosphorylation by CDK”, particularly the amino-acids motifs TP and SP.

The mutated forms of MCM8 proteins obtained by mutations located on a site of phosphorylation by CDKs are either active, either inactive in their helicase function.

According to the invention, the mutated forms of MCM8 are tested as described above for their ability to activate or inhibit DNA replication.

The invention further relates to the use as defined above, wherein said mutations are chosen among the followings:
modification of the conserved threonine (T) in the TP motif to alanine (A) or an equivalent amino acid and modification of the conserved serine (S) in the SP motif to alanine (A) or an equivalent amino acid,
modification of the conserved threonine (T) in the TP motif to glutamate (E) or an equivalent amino acid and modification of the conserved serine (S) in the SP motif to glutamate (E) or an equivalent amino acid.

The present invention also relates to the use as defined above, wherein said mutation is located on a position which is essential for the helicase function of MCM8 protein, and is notably selected from the group composed of:
the nucleotide sequence represented by SEQ ID NO: 23, corresponding to nucleotides 1345-1368 of the xenopus MCM8 gene represented by SEQ ID NO: 1, encoding the amino-acid sequence represented by SEQ ID NO: 24, corresponding to amino acids 449-456 of the xenopus MCM8 protein represented by SEQ ID NO: 2,
the nucleotide sequence represented by SEQ ID NO: 25, corresponding to nucleotides 1537-1548 of the xenopus MCM8 gene represented by SEQ ID NO: 1, encoding the amino-acid sequence represented by SEQ ID NO: 26, corresponding to amino acids 513-516 of the xenopus MCM8 protein represented by SEQ ID NO: 2,
the nucleotide sequence represented by SEQ ID NO: 27, corresponding to nucleotides 1360-1383 of the human MCM8 gene represented by SEQ ID NO: 9 or SEQ ID NO: 15, encoding the amino-acid sequence represented by SEQ ID NO: 28, corresponding to amino acids 454-461 of the human MCM8 protein represented by SEQ ID NO: 10 or SEQ ID NO: 16,
the nucleotide sequence represented by SEQ ID NO: 29, corresponding to nucleotides 1552-1563 of the human MCM8 gene represented by SEQ ID NO: 9 or SEQ ID NO: 15, encoding the amino-acid sequence represented by SEQ ID NO: 30, corresponding to amino acids 518-521 of the human MCM8 protein represented by SEQ ID NO: 10 or SEQ ID NO: 16,
the nucleotide sequence represented by SEQ ID NO: 31, corresponding to nucleotides 1312-1338 of the human MCM8 gene represented by SEQ ID NO: 3 or SEQ ID NO: 13, encoding the amino-acid sequence represented by SEQ ID NO: 32, corresponding to amino acids 438-446 of the human MCM8 protein represented by SEQ ID NO: 4 or SEQ ID NO: 14,
the nucleotide sequence represented by SEQ ID NO: 33, corresponding to nucleotides 1504-1515 of the human MCM8 gene represented by SEQ ID NO: 3 or SEQ ID NO: 13, encoding the amino-acid sequence represented by SEQ ID NO: 34, corresponding to amino acids 502-505 of the human MCM8 protein represented by SEQ ID NO: 4 or SEQ ID NO: 14,
the nucleotide sequence represented by SEQ ID NO: 35, corresponding to nucleotides 1339-1362 of the murine MCM8 gene represented by SEQ ID NO: 17, encoding the amino-acid sequence represented by SEQ ID NO: 36, corresponding to amino acids 447-454 of the murine MCM8 protein represented by SEQ ID NO: 18,
the nucleotide sequence represented by SEQ ID NO: 37, corresponding to nucleotides 1531-1542 of the murine MCM8 gene represented by SEQ ID NO: 17, encoding the amino-acid sequence represented by SEQ ID NO: 38, corresponding to amino acids 511-514 of the murine MCM8 protein represented by SEQ ID NO: 18,
the nucleotide sequence represented by SEQ ID NO: 39, corresponding to nucleotides 1255-1278 of the murine MCM8 gene represented by SEQ ID NO: 19, encoding the amino-acid sequence represented by SEQ ID NO: 40, corresponding to amino acids 419-426 of the murine MCM8 protein represented by SEQ ID NO: 20,
the nucleotide sequence represented by SEQ ID NO: 41, corresponding to nucleotides 1447-1458 of the murine MCM8 gene represented by SEQ ID NO: 19, encoding the amino-acid sequence represented by SEQ ID NO: 42, corresponding to amino acids 483-486 of the murine MCM8 protein represented by SEQ ID NO: 20.

These mutations are located on a position which is essential for the helicase function of MCM8 protein, as they are located on the Walker A motif or on the Walker B motif of the MCM8 gene.

According to the present invention, some mutated forms of the MCM8 protein may lose their helicase function or have an attenuated helicase activity and thus may be used to. decrease the proliferation of cells, in particular of cancer cells.

The invention also relates to the use as defined above, wherein said mutations are chosen among the followings:
modification of the conserved lysine (K) in the Walker A motif GxxGxGK to alanine. (A) or threonine (T) or other non polar or polar neutral amino acids,
modification of the conserved aspartic acid (D) in the Walker B motif DExx to alanine (A) or threonine (T) or other non polar or polar neutral amino acids.

The above modifications of the conserved lysine in the Walker A and/or the conserved aspartic acid in the Walker B lead to mutated forms of the MCM8 which have no helicase activity.

The mutated forms of MCM8 which have no helicase activity may be used in excess by comparison to the native active protein, to decrease the rate of cell proliferation.

The invention further relates to the use of inhibitors of the MCM8 protein to induce the transformation of non tumoral cells into tumoral cells, said inhibitors of the MCM8 protein being chosen among antisense nucleic acids or silencing RNA or antibodies directed against MCM8.

MCM8 is a DNA helicase whose function is required to promote efficient and complete replication of the genome. The Inventors have demonstrated that in the vertebrate Xenopus laevis, the absence of the MCM8 protein causes a slow rate of DNA synthesis and a defect in the retention onto chromatin of DNA polymerase α and the single stranded binding protein RPA34, two key components of the functional unit of DNA synthesis, the replication fork (Maiorano et al., 2005, Cell). The Inventors have also shown that the slow rate of DNA synthesis observed in the absence of MCM8 induces DNA damage, such as double strand breaks (Maiorano, Valentin, and Mechali, unpublished). The production of double strand breaks constitutes a dangerous situation for the cell as these breaks can induce chromosome rearrangements (McGlynn and Loyd, 2002). Therefore, mutations in the MCM8 gene or inhibitors of the MCM8 expression or inhibitors of the MCM8 itself, that lower or eliminate the DNA helicase activity of the MCM8 protein are potential source of DNA damage and therefore genomic instability.

Thus, the inactivation of the MCM8 gene by the human hepatitis virus, which has been observed in patients with liver cancer (Gozuacik et al., 2001), may be a direct consequence of the inactivation of the DNA helicase function of MCM8.

Inactivation of the MCM8 protein can lead in general to the establishment of a cancerous state by directly affecting the structure and the general organization of the genome, by inducing translocation and/or recombination of parts of the chromosomes and can be used to generate new cancer cell lines models. Said cancer cell lines models are useful tools to study the mechanisms of cancer development and to test or screen new drugs for the treatment of cancer.

The invention also relates to a method for the screening of biologically active agents useful in the treatment of human or animal pathology linked to a dysfunction of the expression of the MCM8 gene, said method comprising:
administering a potential agent to a non-human transgenic animal model for MCM8 gene function, particularly chosen among a MCM8 knock-out model and a model of exogenous and stably transmitted MCM8 sequence, and
determining the effect of said agent on the development of the transgenic animal and/or the development of diseases such as those defined above, and in particular the development of cancer.

The term “non-human animal” includes all mammals expect for humans, advantageously rodents and in particular mice.

The term “transgenic animal” denotes an animal into whose genome has been introduced an exogenous gene construct, which has been inserted either randomly into a chromosome, or very specifically at the locus of an endogenous gene.

In a MCM8 knock-out model, the exogenous gene construct has been inserted at the locus of the MCM8 gene, resulting in the impossibility of expressing this MCM8 gene, since it is either interrupted or entirely or partially replaced by a construct such that it no longer allows expression of the endogenous gene, or alternatively a construct which, in addition to the deletion of the endogenous gene, introduces an exogenous gene. Such animals will be referred to as “knock-out” animals or animals in which the abovementioned endogenous gene is invalidated.

A model of exogenous and stably transmitted MCM8 sequence can be obtained by transfection of the cells of the animal (such as stem cells or in vitro cultured cell lines) with a DNA plasmid bearing wild-type or mutated. forms of the MCM8 gene under control of promoter sequence of the MCM8 gene or promoters for standard reporter genes which are constitutively expressed or whose expression can be controlled by induction with inducers of the expression of the above mentioned promoters, integration of such plasmid in the chromosome of such cells so that this transgene is now stably transmitted to the cell progeny.

The effect of the agent is determined by morphological and/or phenotypical analysis of the transgenic animal, and/or by molecular analysis by measure of cell proliferation and/or cell death and/or cell differentiation and/or cell apoptosis, and/or determination of the karyotype of the animal, that is to say analysis of the number and structure of the chromosomes of cells chosen from the whole embryo or tissues of the animal.

The present invention also relates to a method for the in vitro or ex vivo screening of drugs useful in the treatment of human or animal pathology linked to a dysfunction of the expression of the MCM8 gene, said method comprising contacting of the potential drugs with cells such as cancer cells or transformed cells and especially liver, brain, muscle, skin or gut cells wherein a decrease of the expression of the MCM8 helicase is induced by transformation of said cells with recombinant and/or mutated forms of the human or murine or xenopus MCM8 gene, or of parts of said gene, or of transcripts thereof, or of antisense nucleic acids able to hybridize with part of said gene or transcripts, or of silencing RNA derived from parts of said transcripts and able to repress said MCM8 gene, and screening the drugs able to inhibit the proliferation of said transformed cells.

According to another embodiment, the present invention relates to a method for the in vitro or ex vivo screening of drugs useful in the treatment of human or animal pathology linked to a dysfunction of the expression of the MCM8 gene, said method comprising contacting of the potential drugs with cells such as cancer cells or cells wherein recombinant and/or mutated active forms of MCM8 helicase are introduced or transformed cells and especially liver, brain, muscle, skin or gut cells wherein an increase of the expression of an active form of MCM8 helicase is induced by transformation of said cells with recombinant and/or mutated forms of the human or murine or xenopus MCM8 gene, or of parts of said gene, or of transcripts thereof, and screening the drugs able to inhibit the proliferation of said cells.

The expression “active forms of MCM8 helicase” means that the MCM8 proteins have an helicase activity.

In the above embodiment, the term “drugs” refers to inhibitors of DNA replication whose target is the DNA helicase. The inhibitors of DNA replication can be chosen among dibenzothiepin and its analogues, non-hydrolysable NTPs such as γATP, DNA-interacting ligands such as nogalamycin, daunorubicin, ethidium bromide, mitoxantrone, actinomycin, netropsin and cisplatin, 4,5,6,7-tetrabromo-1H-benzotriazole (TBBT), peptides binding DNA that inhibit the unwinding of the double helix by the helicase, bananins and its derivatives, the aminothiazolylphenyl-containing compounds BILS 179 BS and BILS 45 BS, 5′-O-(4-fluorosulphonylbenzoyl)-esters of ribavirin (FSBR), adenosine (FSBA), guanosine (FSBG) and inosine (FSBI), CDKs inhibitors such as staurosporines and its derivatives.

In order to screen potential drugs inhibiting cell proliferation, proliferation tests are carried out on the proliferative cells.

According to another embodiment, the present invention relates to a method for the in vitro or ex vivo screening of drugs useful in the treatment of human or animal pathology linked to a dysfunction of the expression of the MCM8 gene, said method comprising contacting of the potential drugs with transformed cells and especially liver, brain, muscle, skin or gut cells wherein an increase of the expression of an inactive MCM8 helicase is induced by transformation of said cells with recombinant and/or mutated forms of the human or murine or xenopus MCM8 gene, or of parts of said gene, or of transcripts thereof, or wherein a decrease of the expression of the MCM8 helicase is induced by transformation of said cells with antisense nucleic acids able to hybridize with part of said gene or transcripts, or of silencing RNA derived from parts of said transcripts and able to repress said MCM8 gene, and screening the drugs able to stimulate the proliferation of said transformed cells.

In the above embodiment, the term “drugs” refers to activators of DNA replication whose target is the DNA helicase. The activators of DNA replication can be chosen among caffeine, tamoxifen in uterine tissues, leptomycin B, CDKs inhibitors such as staurosporines.

In order to screen potential drugs stimulating cell proliferation, proliferation tests are carried out on the non proliferative cells.

The invention also relates to a method for the in vitro or ex vivo production of catalytically active MCM8 helicase in foreign expression systems, such as insect cells (Sf9) or equivalent or in vitro systems for coupled transcription/translation of the MCM8 cDNA, such as rabbit reticulocytes systems or lysate of E. coli cells or translation of the MCM8 mRNA into xenopus oocytes or egg extracts, under form of a tagged recombinant protein, comprising the steps of:
lysis of cells expressing MCM8 proteins in the following buffer or equivalent, 20 mM TrisHCl pH 8.5, 100 mM KCl, 5 mM β-mercaptoethanol, 5-10 mM imidazole, 10% glycerol (v/v) proteases inhibitors;
purification of the soluble MCM8 proteins by nickel affinity chromatography technology or equivalent or similar affinity chromatography technology;
elution of bound proteins in 10 mM TrisHCl pH 8.5; 100 mM KCl; 5 mM β-mercaptoethanol; 100-250 mM imidazole, 10% glycerol (v/v) proteases inhibitors;
supplementation of purified MCM8 proteins, with or without cleaved tag, with 0.1 mg/ml of BSA;
desaltation on a Bio-spin P30 column (Biorad) equilibrated with 20 mM TrisHCl pH 7.4, 150 mM NaCl, 0.5 mM EDTA, 1 mM DTT, 0.01% Triton X-100 for helicase and ATPase activities, or in XB (100 mM KCl, 0.1 mM CaCl₂, 2 mM MgCl₂, 10 mM Hepes-KOH, 50 mM sucrose, pH 7.7) for egg extracts reconstitution experiments; and
supplementation of the protein with 25% glycerol and storage at −20° C. The rabbit reticulocytes systems and lysate of E. coli cells are ex vivo cell free extracts that can transcribe a given cDNA into mRNA and translate the mRNA into a protein. Such a system may be valuable to produce catalytically active protein to perform in vitro activity assays.

The recombinant proteins are tagged either at the N- or C-terminal with well-known sequence Tag, such as Hist-Tag, Myc-Tag, Flag-Tag, Tap-Tag, GST-tag, MAL-Tag, in order to facilitate the purification of the protein. Preferentially, the sequence tag can be removed by an enzymatic or chemical reaction involving the use of thrombin and/or TEV protease or similar enzymatic activities.

The invention described herein also relates to a DNA vector containing an MCM8 gene and in particular a gene of SEQ ID NO: 1 or SEQ ID NO: 3 or SEQ ID NO: 5 or SEQ ID NO: 7 or SEQ ID NO: 9 or SEQ ID NO: 11 or SEQ ID NO: 13 or SEQ ID NO: 15 or SEQ ID NO: 17 or SEQ ID NO: 19 or SEQ ID NO: 21, or a mutated form of the MCM8 gene as defined above, operatively linked to regulatory sequences.

The term “operably linked” means that the nucleotide sequence is linked to a regulatory sequence in a manner which allows the expression of the nucleic acid sequence. The regulatory sequences are well known by the man skilled in the art. They include promoters, enhancers and other expression control elements.

The invention also provides a host cell transformed with a DNA vector as defined above.

The host cell according to the present invention include prokaryotic host cells (bacterial cells), such as E. coli, Streptomyces, Pseudomonas, Serratia marcescens and salmonella typhimurium or eukaryotic cells such as insect cells, in particular baculovirus-infected Sft9 cells, or fungal cells, such as yeast cells, or plant cells or mammalian cells.

The invention further relates to a recombinant protein obtained by the expression of the DNA vector as defined above.

The DNA vector containing the MCM8 gene as defined above is used to produce a recombinant form of the protein by recombinant technology. Recombinant technology comprises the steps of ligating the nucleotide sequence into a gene construct such as an expression vector and transforming or transfecting said gene construct into host cells. The host cells that express the protein are then lysed and the recombinant protein in isolated and purified, for example by chromatography.

The present invention relates to an antibody or antigen-binding fragment which binds to an MCM8 protein or part of an MCM8 protein or to a modified active MCM8 protein or to a modified part of an MCM8 protein, and in particular to polypeptides comprising the totality or part of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ ID NO: 22.

The antibody can be polyclonal or monoclonal and the term “antibody” is intended to encompass both polyclonal and monoclonal antibodies. The terms “polyclonal” and “monoclonal” refer to the degree of homogeneity of an antibody preparation, and are not intended to be limited to a particular method of production.

The present invention relates to antibodies which bind to MCM8 protein or part of an MCM8 protein, or to a mutated form of the MCM8 protein or part thereof. A mammal, such as a rabbit, a mouse or a hamster, can be immunized with an immunogenic form of the protein, such as the entire protein or a part of it. The protein or part of it can be administered in the presence of an adjuvant.

The term “immunogenic” refers to the ability of a molecule to elicit an antibody response. Techniques for conferring immunogenicity to a protein or part of it which is not itself immunogenic include conjugation to carriers or other techniques well known in the art.

The immunization process can be monitored by detection of antibody titers in plasma or serum. Standard immunoassays, such as ELISA can be used with the immunogenic protein or peptide as antigen to assess the levels of antibody.

The invention relates in particular to monoclonal and polyclonal antibodies directed against an MCM8. helicase or against polypeptides comprising part of an MCM8 helicase and in particular against polypeptides comprising the totality or part of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO:20 or SEQ ID NO: 22.

According to another embodiment, the invention relates to pharmaceutical preparations comprising an MCM8 helicase or a polypeptide comprising part of an MCM8 helicase and in particular a polypeptide comprising the totality or part of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ D NO: 22 or a mutated form of the MCM8 helicase as defined above.

The pharmaceutical preparation of the present invention can be formulated with a physiologically acceptable medium, such as water, buffered saline, polyols (glycerol, propylene glycol, liquid polyethylene glycol) or dextrose solutions. Preferentially, the pharmaceutical preparations is formulated in a vector which will allow the delivery of said preparation inside the target cells. The pharmaceutical preparation can be administered by intradermal, intramuscular, intraperitoneal, intravenous, subcutaneous or oral way.

The pharmaceutical preparation may also be administered as part of a combinatorial therapy with other agents, such as inhibitors or activators of cell proliferation. Inhibitors of cell proliferation can be chosen among aphidicoline, cis-platinum, etoposides, lovastatin, mimosine, nocodazole. Activators of cell proliferation can be chosen among growth factors such as EGF (Epidermal Growth Factor), FGF (Fibroblast Growth Factor), NGF (Nerve Growth Factor) and analogues, and lipopolysaccharides.

The invention also relates to humanized immunoglobulin chains having specificity for an MCM8 helicase and in particular for polypeptides of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ ID NO: 22.

The term “humanized immunoglobulin chains” refers to human immunoglobulins produced for example in mouse.

The invention further relates to a method for inhibiting cell proliferation or allowing a better replication of the DNA, comprising administering an agonist or antagonist of an MCM8 helicase in a way that the agonist or antagonist enters the cell, said antagonist causing the inhibition of DNA replication and said agonist contributing to the restoration of cell replication or to the ability of the cell to replicate DNA in unfavorable conditions.

The invention relates in particular to a method for inhibiting cell proliferation or allowing a better replication of the DNA in vitro or ex vivo, comprising administering an agonist or antagonist of an MCM8 helicase in a way that the agonist or antagonist enters the cell, said antagonist causing the inhibition of DNA replication and said agonist contributing to the restoration of cell replication or to the ability of the cell to replicate DNA in unfavorable conditions.

### DESCRIPTION OF THE FIGURES

FIG. 1A, FIG. 1B, FIG. 1C and FIG. 1D

MCM8 is an MCM2-7-like protein that does not associate with MCM2-7 in egg cytosol.

FIG. 1A: Features of Xenopus MCM8 protein. Numbers indicate amino-acids.

FIG. 1B: Characterization of the MCM8 antibody. Autoradiography of in vitro [35]-labelled proteins (lanes 1, 2) obtained by coupled transcription-translation of the MCM8 cDNA in the sense (lane 1) or antisense (lane 2) orientation. Translation products were also probed with the MCM8 antibody by western blot (lanes 3, 4).

FIG. 1C: Western blot of Xenopus egg extracts with pre-immune (lane 1) or MCM8-specific serum (MCM8, lane 2), raised against recombinant MCM8.

FIG. 1D: MCM8 does not associate with MCM2-7 proteins in S phase egg extracts. Western blot of proteins immunoprecipitated with MCM3 and revealed with an anti-MCM8 antibody (lane 1) or with an antibody raised against an epitope conserved in all MCM2-8 proteins (lane 2). IgGs correspond to immunoglobulins.

FIG. 2A, FIG. 2B and FIG. 2C

MCM8 binds chromatin at the onset of DNA synthesis.

FIG. 2A: Dynamics of Cdt1, MCM2, MCM8, PCNA and Ccd45 chromatin binding during S phase. Western blot of detergent-resistant chromatin fractions (lanes 4-11) obtained by incubation of sperm nuclei in Xenopus S-phase egg extracts and isolated at the indicated times. A sample of egg cytoplasm (1 μl, lane 1), demembranated sperm nuclei (25,000; lane 2) or insoluble material obtained by centrifugation of egg cytoplasm (lane 3) were also included as controls.

FIG. 2B: MCM8 (circles) binds to chromatin at the beginning of DNA synthesis at a time when MCM2 (squares) and Cdt1(diamonds) are displaced. DNA synthesis (bars) was measured at the indicated times by incorporation of α-[³²P] dCTP as described in experimental procedures. Western blot signals obtained with Cdt1, MCM2 and MCM8 antibodies in FIG. 2A were quantified and plotted as percent of chromatin-bound proteins compared to their maximal level obtained during S phase. The quantification graph obtained was superimposed with that of DNA synthesis.

FIG. 2C: MCM8 does not bind to chromatin in membrane-depleted egg extracts. Sperm chromatin was incubated in “high speed” extracts for 60 minutes and chromatin was isolated as described in experimental procedures in the presence of 0.1% NP-40. Cytosolic (Cyto) and chromatin (Chr) fractions were analyzed by western blot with MCM3 and MCM8 antibodies.

FIG. 3A, FIG. 3B, FIG. 3C and FIG. 3D

Binding and distribution of MCM8 on chromatin during S-phase

FIG. 3A: Punctuate distribution of MCM8 on chromatin. Detergent-extracted nuclei formed in egg extracts were isolated during early (30 minutes), mid (60 minutes), or late (90 minutes) S phase. Nuclei were stained with Hoechst to visualize DNA and with MCM8 and MCM3 antibodies. Nuclei were also isolated after sixty minutes incubation in egg extracts treated with geminin (gem).

FIG. 3B: Punctuate distribution of MCM8 on chromatin. Detergent-extracted nuclei were also isolated after sixty minutes incubation in egg extracts treated with p21 or aphidicolin. Aphidicolin or p21 were added either before initiation of DNA synthesis (t=0 min; I), or during elongation (t=50 min; E). Nuclei were stained with Hoechst to visualize DNA and with MCM8 and MCM3 antibodies.

FIG. 3C: Binding of MCM8 to chromatin in the presence of aphidicolin. Western blot of chromatin fractions formed in the absence (control) or presence (+aphi) of aphidicolin (50 μg/ml). Aphidicolin was added at time zero in Xenopus egg extracts. Chromatin fractions were prepared after 60 or 120 minutes incubation (aphi at initiation). Proteins were detected with the DNA polα, MCM8, PCNA and ORC2 antibodies.

FIG. 3D: Binding of MCM8 to chromatin in the presence of aphidicolin. Western blot of chromatin fractions formed in the absence (−) or presence (+aphi) of aphidicolin (50 μg/ml). Aphidicolin was added after 50 minutes incubation in Xenopus egg extracts. Chromatin fractions were prepared 30 min after addition of aphidicolin during elongation. Proteins were detected with the DNA polα and MCM8 antibodies.

FIG. 4A, FIG. 34B, FIG. 4C and FIG. 4D

MCM8 is required for efficient DNA synthesis

FIG. 4A: Depletion of MCM8 does not remove MCM2-7 proteins, nor ORC1. Western blot of S-phase egg extracts depleted with either mock (first column) or MCM8 antibodies (second column). Depletion of MCM8 was 99% as judged by scanning and quantification of the western blot signals. MCM2-8 proteins were revealed with an antibody raised against a motif conserved in this protein family (MCM pep, Maiorano et al., 2000a). Numbers on the right hand side of the panel indicate MCM2-8 proteins. Stars indicate the mobility of MCM8 polypeptides recognized by the anti-peptide antibody.

FIG. 4B: Purification of MCM8 from Xenopus egg cytoplasm. Silver stain of the MCM8 protein immunopurified from egg extracts (lane 1). Western blot of the purified MCM8 protein with the MCM8 antibody (lane 2).

FIG. 4C: MCM8 is required for efficient DNA synthesis. Either mock-depleted or MCM8-depleted S-phase egg extracts were incubated with sperm chromatin (3 ng/μl) and total DNA synthesis was measured as in FIG. 2B after 150 minutes incubation. The amount of DNA synthesized in MCM8-depleted extracts in three independent experiments (Dep I-III), and that synthesized in MCM8-depleted extracts reconstituted with Xenopus MCM8 protein (+MCM8) is shown.

FIG. 4D: MCM8 is not required for nuclear assembly. Nuclei formed in either mock-depleted or MCM8-depleted extracts were observed by phase contrast (phase) or fluorescence microscopy (DNA). DNA was visualized by staining with Hoechst.

FIG. 5A, FIG. 5B, FIG. 5C, FIG. 5D, FIG. 5E and FIG. 5F

Slow rate of DNA synthesis in MCM8-depleted extracts

FIG. 5A: MCM8 is required for processive DNA synthesis. Kinetics of chromosomal DNA synthesis (sperm chromatin, 3 ng/μl) in S-phase egg extracts mock-depleted (squares), MCM8-depleted (circles), or MCM3-depleted (diamonds). DNA synthesis was measured as in FIG. 2B. A western blot of egg extracts mock-depleted (lane 1), MCM8-depleted (lane 2) or MCM3-depleted (lane 3) probed with the MCM8 and MCM3 antibodies is shown in the inset. Depletion of MCM8 was 99%.

FIG. 5B: MCM8 is not required for replication of single-stranded DNA templates. Kinetics of replication of single-stranded M13 DNA (10 ng/μl) in mock-depleted (squares) or MCM8-depleted (diamonds) extracts. DNA synthesis was measured as in FIG. 2B.

FIG. 5C: Nascent DNA accumulates in MCM8-depleted extracts. Autoradiography of α-[³²P] dCTP-labelled DNA synthesized in mock-depleted, MCM8-depleted, or in mock-depleted extracts in the presence of either 10 μg/ml of aphidicolin (Aphi), or 100 nM of geminin protein. Total DNA was extracted at the indicated time during S phase (FIG. 5A) and analyzed by alkaline agarose gel electrophoresis. Standard DNA molecular weight markers (kb) were run in parallel.

FIG. 5D: Densitometry scan of replication intermediates observed in either mock-depleted or MCM8-depleted egg extracts at the 90 minutes time point. A line was placed vertically through the middle of lanes 3 (Mock-depleted) or lane 6 (MCM8-depleted) of FIG. 5C. The intensity of the radioactive signals was measured, normalized and plotted as function of the distance from the origin of migration of the samples.

FIG. 5E: Overexposure of DNA synthesis products at early time points of panel of FIG. 5C.

FIG. 5F: The incorporation of the nucleotide analogue biotine-dUTP (Replication) was observed by indirect immunofluorescence on nuclei assembled in either mock-depleted or MCM8-depleted extracts at the 120 minutes time point. DNA was visualized by Hoechst staining.

FIG. 6A, FIG. 6B, FIG. 6C, FIG. 6D, FIG. 6E and FIG. 6F

MCM8 is a DNA helicase that regulates the recruitment of RPA34 and DNA polymerase α on replicating chromatin

FIG. 6A: Purification of recombinant MCM8. Wild-type (lane 1) and a mutant form of MCM8 in the ATP binding site (lane 2), were expressed and purified from Sf9 cells by nickel chromatography. One aliquot of the purified protein was analyzed by SDS-PAGE followed by staining with Coomassie Blue.

FIG. 6B: DNA helicase activity of MCM8. Displacement of a branched 40-mer oligonucleotide labelled with [³²P] ATP and annealed to ssM13 DNA (origin) as determined by autoradiography following acrylamide gel electrophoresis. The annealed substrate was incubated at room temperature for 1 hour with 25 ng (lane 1) or 50 ng (lanes 2-5) of recombinant MCM8, in the presence or absence of 10 mM of the indicated substrates (lanes 3-5). The displacement activity of 50 ng of BSA (lane 6) and the displacement of the annealed substrate by heat denaturation (boiled, lane 7) are also shown.

FIG. 6C: DNA helicase activity of MCM8 requires an intact ATP binding site and is not stimulated by the MCM2-7 complex. Oligonucleotide displacement activity of recombinant MCM8 alone (lanes 3-4, 15 and 30 ng respectively) or that of MCM8 (30 ng) in combination with 100 ng of MCM2-7 complex (lane 5). The helicase activity of recombinant MCM8 mutated in the ATP binding site (lane 2, 75 ng) and that of the MCM2-7 complex alone (lane 6, 100 ng) are also shown. The displacement activity of 50 ng of BSA (lane 1) and the displacement of the annealed substrate by heat denaturation (boiled, lane 7) are also shown.

FIG. 6D: MCM8 displays DNA-dependent ATPase activity. Autoradiography of a thin layer chromatography, of reactions carried out in the presence of γ-[32P] ATP. The position of released ³²P is indicated (Pi) as well as that of the origin of migration (Origin). Reactions were carried out with 15 ng (lane 3) or 30 ng (lane 4) of MCM8 in the presence of ssDNA, or without DNA (lane 2) with 30 ng of MCM8. The ATPase activity of MCM8 mutated in the ATP binding site (60 ng, lane 5) and that of BSA (100 ng, lane 1) are also shown.

FIG. 6E: MCM8 mutated in the ATP binding site does not rescue DNA synthesis in MCM8-depleted extracts. Replication of sperm chromatin in either mock-depleted (1) or MCM8-depleted (2-4) extracts rescued with wild-type MCM8 (WT, lane 3, 30 ng) or MCM8 mutated in the ATP binding site (KA, lane 4, 50 ng). DNA synthesis was measured after 150 minutes incubation.

FIG. 6F: Poor recruitment of RPA and DNA polymerase a onto chromatin in MCM8-depleted extracts. Western blot of detergent-resistant chromatin fractions (lanes 1, 2) or egg supernatants (lanes 3, 4) obtained in mock-depleted or MCM8-depleted extracts. Chromatin was isolated after 60 minutes incubation in extracts and analyzed with the MCM3, RPA34 and DNA polα antibodies.

FIG. 7A and FIG. 7B

MCM8 is confined to replication factories

FIG. 7A: Distribution of MCM8, RPA34 and replication foci (biotin-dUTP) on sperm nuclei in early or mid S phase. Nuclei were detergent-extracted and stained with RPA34 or MCM8 antibodies. Replication foci (biotin-dUTP) were labelled by a short pulse of biotin dUTP and revealed with streptavidin. Merge of the signals (MCM8/Biotin and RPA/biotin) is also shown. DNA is detected by staining with DAPI. A continuous arrow indicates replicating foci whereas a dashed arrows indicates RPA foci on pre-replicating chromatin. Magnifications of the staining pattern of MCM8, RPA34 and that of replication foci (biotin-dUTP) during S phase are also shown as insets to increase the resolution.

FIG. 7B: MCM8 and RPA34 co-localize on chromatin. Nuclei formed in egg extracts after 60 minutes incubation in the presence of 10 μg/ml of aphidicolin were co-stained with MCM8 and RPA34 antibodies. Merge of the two signals is also shown (MCM8/RPA). DNA is revealed by staining with DAPI.

FIG. 8

MCM8 is not required for nuclear growth

Nuclear growth is not affected in MCM8-depleted extracts. Nuclei formed in either mock-depleted (Δ-Mock) or MCM8-depleted (Δ-Mcm8) egg extracts were stained with Hoechst, fixed with paraformaldehyde and observed by both fluorescence microscopy (Hoechst) or phase contrast. Depletion of MCM8 was over 99% as judged by quantification of western blot signals obtained with MCM8 antibodies. About 100 nuclei at each time point were observed and the mean value of nuclear diameter at the 120 minutes time point is indicated (μm).

FIG. 9A, FIG. 9B, FIG. 9C, FIG. 9D and FIG. 9E

MCM8 is required for efficient replication of chromosomal DNA

FIG. 9A: Slow DNA replication in the absence of MCM8. Kinetics of DNA synthesis of egg extracts double-depleted with the indicated antibodies coupled to recombinant protein A. Depletion was more than 99% as judged by scanning and quantification of the western blot signals of MCM8-depleted egg extracts (insert, lane 2) compared to mock-depleted extracts (lane 1). MCM8-depleted egg extracts were also rescued by addition of wild-type, recombinant MCM8.

FIG. 9B: Depletion of MCM8 does not remove RPA34 from egg extracts. Western blot of mock-depleted (lane 1) or MCM8-depleted (lane 2) egg supernatants probed with the MCM8 and RPA34 antibodies.

FIG. 9C: Nascent DNA chains accumulate in MCM8-depleted egg extracts. The products of DNA synthesis of the reactions described in FIG. 9A were analyzed by alkaline gel electrophoresis. Mock-depleted (lane 1), MCM8-depleted (lane 2), and MCM8-depleted reactions reconstituted with recombinant MCM8 (lane 3) are shown.

FIG. 9D: MCM8 does not form a complex with RPA34 in egg cytoplasm. Western blot of immunoprecipitates (P) obtained from egg extracts incubated with control antibodies (Mock, lane 2) or MCM8 antibodies (MCM8, lane 3). RPA34 was revealed with a specific monoclonal antibody (lane 1).

FIG. 9E: MCM8 is dispensable for DNA unwinding at the initiation step. Western blot of chromatin fractions obtained from the depletion experiment described in FIGS. 9B and 9C, and isolated from either mock-depleted (lane 1-2) or MCM8-depleted egg extracts (lane 3) in the absence (lane 1) or the presence (lanes 2-3) of 50 μg/ml of aphidicolin. Proteins were revealed with the RPA34 and ORC1 specific antibodies.

### EXAMPLES

The inventors have identified a Xenopus homolog of MCM8 and characterized its function using in vitro cell-free extracts. They show that MCM8 binds chromatin after licensing, only when DNA synthesis is initiated. Unlike MCM2-7, MCM8 co-localizes with RPA34 and DNA replication foci on replicating chromatin MCM8 is required for efficient progression of replication forks suggesting a role in DNA unwinding. Both ATPase and helicase activities are associated with recombinant MCM8 in vitro. Mutation in the ATP binding site of MCM8 abolishes both activities and cannot complement loss of MCM8.

These results strongly suggest that MCM8 is a specialized MCM2-7-like protein not required for licensing but that specifically functions as a DNA helicase in vivo, regulating progression of replication forks at replication factories.

### Experimental Procedures

### Cloning Procedures

A cDNA coding the amino-terminal of the MCM8 protein was identified by PCR using an MCM2-7 signature-specific primer and a primer specific for a cDNA library (λgt10 cloning vector) made from Xenopus oocytes (Rebagliati et al., 1985). The complete MCM8 cDNA (EMBL accession number AJ867218) was identified in the database as the EST BU906538. For expression of MCM8 in baculovirus-infected Sf9 cells (Bac-to-Bac system, GIBCO), the Xenopus MCM8 cDNA was amplified by PCR and sub-cloned in pFastBacHTb. The MCM8 K⁴⁵⁵ to A⁴⁵⁵ mutant was made using the Quik-change kit (Stratagene).

### Proteins Expression and Purification

An amino-terminal portion of the MCM8 protein (aa 24-402) was expressed in E. coli BL21(λDE3) strain by sub-cloning into the bacterial expression vector PRSET_{B} (Invitrogen). The corresponding recombinant protein was expressed by induction with 1 mM IPTG at 37° C. for 3 hours. Inclusion bodies were prepared and solubilized with 8M Urea. The recombinant protein was purified to homogeneity on a nickel column under denaturing conditions following the supplier instructions (Qiagen). Purified protein was re-natured in vitro as described (Vuillard et al., 1998), dialyzed and concentrated in Centricon-30 (Amicon), and stored at −20° C. Full length MCM8 was transcribed and translated in vitro in rabbit reticulocytes (TNT, Promega) in the presence of ³⁵S-methionine.

Sf9 cells expressing MCM8 proteins were grown for 52 hours at room temperature, harvested, washed in PBS and frozen as a pellet at −80° C. Cells were thawed and lysed following the instructions of the supplier. Soluble MCM8 protein was purified by nickel affinity chromatography. Bound proteins were recovered in the following elution buffer: 10 mM TrisHCl pH 8.5; 100 mM KCl; 5 mM β-mercaptoethanol; 100 mM imidazole, 10% glycerol (v/v) proteases inhibitors (leupeptine, pepstatine and aprotinin, 10 μg/ml each). Purified MCM8 protein was supplemented with 0.1 mg/ml of BSA and desalted on a Bio-spin P30 column (Biorad) equilibrated with 20 mM TrisHCl pH 7.4; 150 mM NaCl; 0.5 mM EDTA; 1 mM DTT; 0.01% Triton X-100 for helicase and ATPase activities, or in XB (100 mM KCl, 0.1 mM CaCl₂, 2 mM MgCl₂, 10 mM Hepes-KOH, 50 mM sucrose, pH 7.7) for reconstitution experiments. Protein was supplemented with 25% glycerol and stored at −20° C.

Xenopus Geminin Δ was expressed in E. coli and purified to homogeneity as previously described (Maiorano et al., 2004; McGarry and Kirschner, 1998). GSTp21 was purified as previously described (Jackson et al., 1995).

### Antibodies

The MCM8 antibody was raised in rabbits using Xenopus MCM8 N-ter protein, and affinity purified by incubation of crude serum on a nitrocellulose membrane saturated with recombinant N-ter MCM8 as described (Adachi and Yanagida, 1989). The anti-MCM2-8 anti-peptide and Cdt1 antibodies have been previously described (Maiorano et al., 2000a; Maiorano et al., 2000b). The anti-MCM2 antibody was a gift of Dr. Ivan Todorov (Todorov et al., 1995). The PCNA antibody has been previously described (Leibovici et al., 1992). The anti-ORC1 antibody was a gift from Dr. J. Blow (Rowles et al., 1999). Antibodies against ORC2 and Cdc45 were provided by Dr. J. Walter (Walter and Newport, 2000). The DNA Pol-α antibody was a gift of Dr. F. Grosse (Max Planck Institute, Germany). The Xenopus RPA34 antibody was as described (Françon et al., 2004).

### Xenopus Egg Extracts and DNA Replication Reactions

Egg extracts were prepared and used as previously described (Mechali and Harland, 1982; Menut et al., 1988). Depletion and reconstitution experiments were as previously described (Maiorano et al., 2000b). Briefly, Xenopus low speed egg extracts were supplemented with cycloheximide (250 μg/ml) and double-depleted with anti-MCM8 serum coupled to Protein-A sepharose beads or recombinant protein A sepharose (Pharmacia, 50% beads to extract ratio), for 40 minutes at 4° C. DNA replication was measured by addition of α-[³²P] dCTP and sperm nuclei (3 ng/μl). For pulse-labelling experiments, nuclei were pulse-labelled for 30 seconds with bio-dUTP (40 μM). Where required, aphidicolin (20 mg/ml in DMSO) was diluted 10-fold in water and supplemented to the reactions at the indicated concentration.

### Immunoprecipitation and Immunopurification Procedures

Immunoprecipitation from egg extracts was performed by diluting the extract 5 times in PBS in the presence of proteases inhibitors (leupeptin, aprotinin and pepstatine, 10 μg/ml each) and incubation with specific antibodies coupled to either protein A or protein G beads (Roche) for 1 hour at 4° C. on a rotating wheel. Beads were washed several times with PBS supplemented with proteases inhibitors and proteins were eluted in Laemmli buffer and analyzed by SDS-PAGE.

Xenopus MCM8 protein was immunopurified from egg extracts with anti-MCM8 serum coupled to high affinity recombinant Protein A-Sepharose (Pharmacia). All buffers were supplemented with proteases inhibitors. Egg extracts were incubated with the MCM8 antibody coupled to Protein A beads (1:3 beads to extract ratio) saturated with 0.5 mg/ml of O BSA, for 1 hour at 4° C. Beads were washed 5 times with 10 volumes of XB (100 mM KCl, 0.1 mM CaCl₂, 2 mM MgCl₂, 10 mM Hepes-KOH, 50 mM sucrose, pH 7.7), once with XB/0.2M NaCl, and the MCM8 protein was finally eluted with 2 volumes of XB/0.8M NaCl for 10 minutes on ice. Eluates were supplemented with 0.05 mg/ml BSA, concentrated at about 1 mg/ml and dialysed against XB/10% glycerol by centrifugation in a microcon-30 (Millipore). Proteins were stored at −20° C.

ATPase and DNA Helicase Assay

ATPase activity of MCM8 proteins was determined as previously described (Ishimi, 1997). Reactions (20 μl) were carried out at 23° C. for 1 hour in the presence or absence of 500 ng of heat-denatured ssM13 DNA. 0.5 μl of each sample was spotted on a cellulose F paper (Merck) and separated by thin layer chromatography as described (Ishimi, 1997). Papers were air dried and exposed to a PhosphorImager screen (Molecular Dynamics). DNA helicase activity was assayed using as substrate single-stranded M13 DNA (Biolabs) annealed to a 40-mer branched oligonucleotide as previously described (Lee and Hurwitz, 2001). Five femtomoles of annealed substrate were incubated with recombinant MCM8 in a reaction of 20 μl and incubated at room temperature for 1 hour. Reaction was stopped by addition of 0.1% SDS, 10 mM EDTA and separated on a 12% acrylamide gel in TBE 1×. Gels were dried and exposed to a PhosphorImager screen (Molecular Dynamics).

DNA helicase activity of recombinant MCM8 can also be assayed using as a substrate single-stranded M13 DNA (Biolabs) annealed by standard procedures (Sambrook et al., 1991) to an oligonucleotide containing 37 bases complementary to M13 DNA and a 40 bases non-complementary tail, in “helicase buffer” (20 mM trisHCl, pH 7.5; 10 mM MgCl2; 0.1 M NaCl; 1 mM DTT). Five femtomoles of annealed substrate are incubated with recombinant MCM8 in a reaction of 20 μl containing 50 mM TrisHCl pH 7.9; 1 mM DTT; 10 mM ATP; 0.5 mg/ml BSA; 10 mM Mg(CH₃COOH)₂, and incubated at room temperature for 1 hour. The DNA helicase activity of MCM8 is very likely to be stimulated by the presence of accessory proteins, such as the single-stranded DNA binding trimeric protein complex RPA, PCNA, RF-C and DNA polymerases. The ATPase activity of MCM8 proteins is carried out in a reactions of 20 μl in helicase buffer at 23° C. for 1 hour in the presence of 500 ng of heat-denatured ssM13 DNA.

### Chromatin Purification and Indirect Immunofluorescence Microscopy

The protocol for chromatin purification has been previously described (Coué et al., 1996; Maiorano et al., 2000a). For immunofluorescence microscopy, at each indicated time point, nuclei were diluted 10 times in XB/0.3% Triton X-100, incubated at room temperature for 15 minutes and fixed with 0.8% of fresh formaldehyde for 5 minutes on ice. Nuclei were then isolated by centrifugation through a 30% glycerol cushion made in XB on a coverslip at 4° C. by centrifugation at 1,500 g and immediately saturated in PBS/BSA 1% at room temperature for 1 hour. Primary antibodies were incubated over night at 4° C. in a wet atmosphere. Biotin dUTP (Roche) was revealed by staining with anti-streptavidin antibodies coupled to Texas Red.

### Alkaline Gel Electrophoresis

Samples obtained from replication reactions were incubated with 0.4 mg/ml of proteinase K for 1 hour at 37° C., extracted with phenol/chloroform and loaded onto a 1.2% agarose alkaline gel (30 mM NaOH, 2.5 mM EDTA). Gels were run over night at 3V/cm with a buffer recirculation system at 4° C. After run gels were fixed for 10 minutes in 7% TCA at room temperature, then dried and exposed to a PhosphorImager screen (Molecular Dynamics).

### Example 1

### Identification of Xenopus MCM8, an MCM2-7-like Protein That is Not Associated With the Soluble MCM2-7 Complex

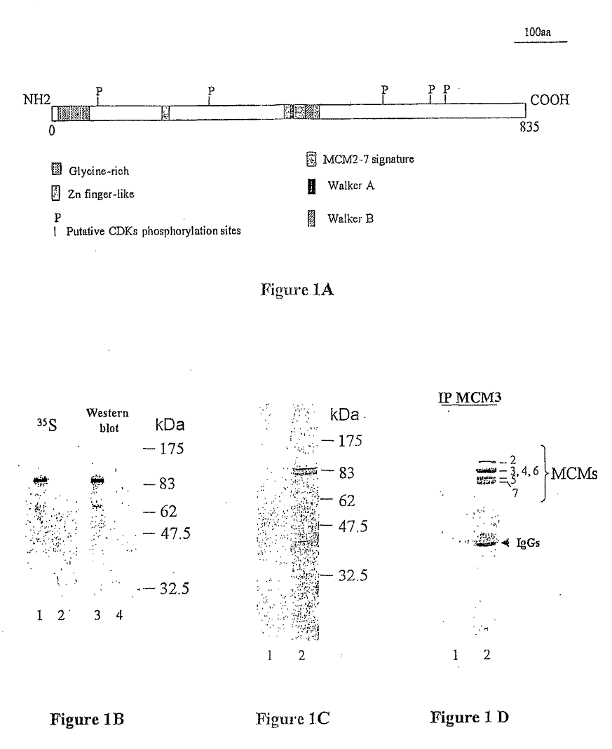

In a PCR-based approach aimed at identifying MCM2-7-related genes (see Experimental procedures), the Inventors have isolated a cDNA that could potentially encode a protein similar to MCM2-7 (average 23%). However, database search shows that the highest homology is obtained with HMCM8 (74% identity), a member of the MCM2-7 protein family recently identified in human cells (Gozuacik et al., 2003). Xenopus MCM8 is highly related to human MCM8 throughout the sequence except 60 amino-acids in the N-terminal, which are arginine- and especially glycine-rich in both proteins (FIG. 1A). A similar glycine-rich region is present in the N-terminus of the Xenopus RPA34 protein.

The predicted MCM8 protein (92.48 kDa) shows similar features to MCM2-7, including a Zn finger-like motif and Walker A and B motifs implicated in the helicase activity of MCM2-7 (FIG. 1A). Interestingly, the Walker A motif of Xenopus and mammalian MCM8 homologs (Gozuacik et al., 2003; Johnson et al., 2003) is a canonical consensus sequence (GxxGxGKS/T), while the one found in MCM2-7 proteins is a deviant consensus in which the third conserved glycine is replaced by either an alanine or serine. This consensus sequence is also observed in the unique MCM2-7-like protein of the archaebacteria M. termoautotrophicum (Kearsey and Labib, 1998). In this respect, MCM8 resembles more to a bona fide helicase than MCM2-7 proteins. Xenopus MCM8 contains five potential phosphorylation sites for Cyclin-Dependent Kinases (CDKs, consensus S/T-P), although none of them is a CDK1/Cyclin B consensus site. Three of these sites (two in the amino- and one in the carboxy-terminal) are conserved in the human MCM8 protein.

The Inventors raised an antibody against the N-terminal part of MCM8, which is not conserved amongst MCM2-7 proteins (less than 9% identity), to avoid cross-reactions with members of the MCM2-7 protein family. The MCM8 antibody specifically recognized the MCM8 protein translated in vitro (FIG. 1B, lane 1 and 3) and a 90 kDa polypeptide, often seen as a doublet, in Xenopus egg extracts (FIG. 1C, lane 2). The MCM8 antibody did not recognize any proteins in MCM3 immunoprecipitates (FIG. 1D, lane 1), which contain the whole MCM2-7 protein complex (FIG. 1D, lane 2, and Maiorano et al., 2000a). The Inventors conclude that MCM8 does not form complexes with MCM2-7 proteins in egg cytosol.

### Example 2

### MCM8 Binds to Chromatin After Licensing, at the Time of Initiation of DNA Synthesis

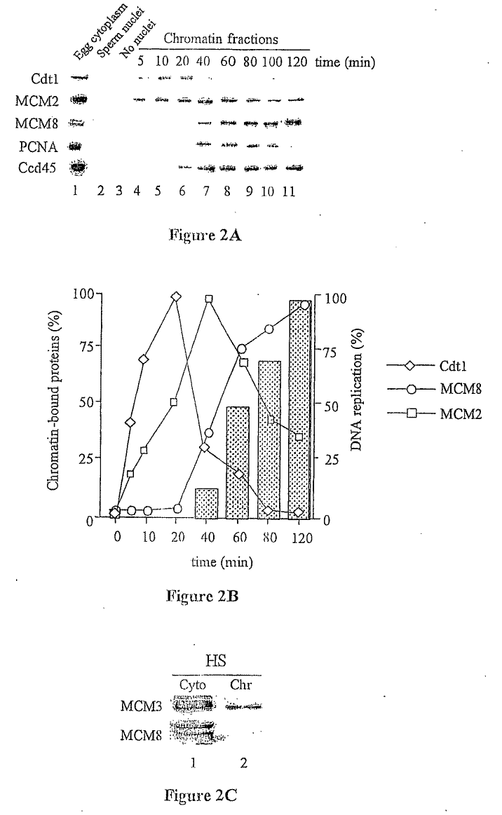

The Inventors have first determined the timing of MCM8 chromatin binding using Xenopus egg extracts synchronized in S-phase and reconstituted with demembranated sperm nuclei (Blow and Laskey, 1986). Detergent-resistant chromatin fractions were isolated and analyzed by western blot (FIG. 2A). DNA synthesis was measured in parallel by incorporation of a radioactive DNA precursor (FIG. 2B, bars). MCM2 binds to sperm chromatin very early (within 5 minutes, lane 4), at the same time as the MCM2-7 loading factor Cdt1, but before initiation of DNA synthesis (FIG. 2B). MCM8 binds to chromatin much later than MCM2 (FIG. 2A, compare lanes 4 and 7), similar to HMCM8 (Gozuacik et al., 2003). By that time the MCM2-7 loading factor Cdt1 began to be removed. Interestingly, binding of MCM8 to chromatin was first observed following accumulation of MCM2-7 proteins onto chromatin (the licensing reaction), after binding of Cdc45, and at the onset of DNA synthesis (FIG. 2B). These results show that, unlike MCM2-7, MCM8 is not recruited to chromatin during formation of pre-replication and pre-initiation complexes (5-20 minutes in the experiment shown), suggesting that MCM8 may not be required for licensing. Maximal level of chromatin-bound MCM8 (FIG. 2A, lanes 8-11) were observed during processive DNA synthesis (FIG. 2B, 60-120 minutes). The timing of MCM8 chromatin binding overlapped with that of PCNA, a DNA polymerase 8 processivity factor required during elongation of DNA synthesis (Waga, 1994). Interestingly, while MCM2 was gradually removed from chromatin during ongoing DNA synthesis as expected (FIG. 2A, lanes 8-11 and FIG. 2B), MCM8 chromatin binding increased slightly and remained constant until completion of S phase, while PCNA dissociated. Quantification of the signals obtained by western blot (FIG. 2B) confirmed the correlation between both Cdt1and MCM2 displacement, and MCM8 chromatin binding. The Inventors conclude that MCM8 binds to chromatin around the time of initiation of DNA synthesis or just afterwards, suggesting a function during this step.

To determine whether initiation of DNA synthesis is required for the binding of MCM8 to chromatin, the Inventors have analyzed the association of MCM8 with chromatin formed in membrane-depleted egg extracts. These extracts are competent to form pre-replication complexes (Coleman et al., 1996; Coue et al., 1998; Coué et al., 1996), but DNA synthesis cannot initiate as Cdc45 and DNA polyrnerases are not loaded (Mimura and Takisawa, 1998). As expected, MCM3 bound to chromatin in these extracts (FIG. 2C, lane 2), while MCM8 did not, although it was detected in the extract (lane 1). This result confirms that MCM8 is not a component of pre-replication complexes and further demonstrates that the binding of MCM8 to chromatin occurs after loading of MCM2-7 proteins.

### Example 3

MCM8 Chromatin Binding Depends Upon MCM2-7 and is Sensitive to the S-CDK Inhibitor p21

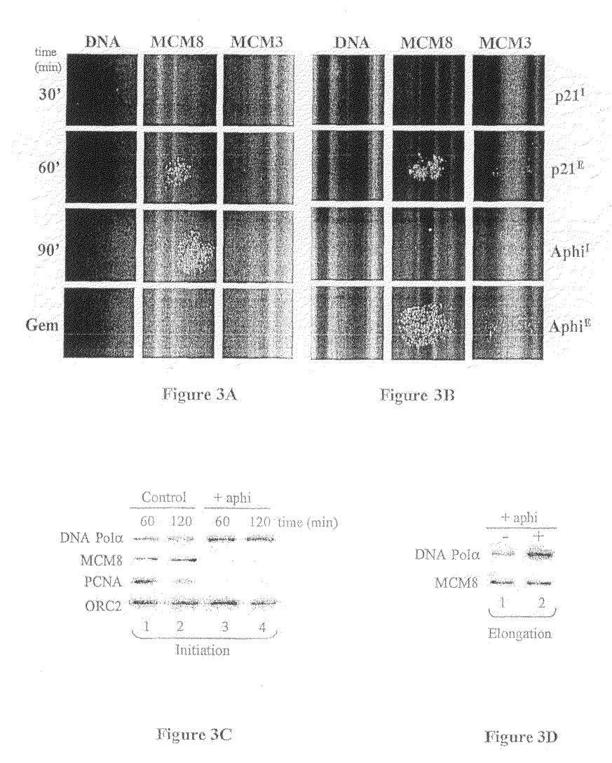

Detergent-extracted nuclei formed in egg extracts and isolated during S-phase were observed by indirect immunofluorescence following staining with both MCM3 and MCM8 antibodies (FIG. 3A). Before initiation of DNA synthesis (30 minutes), MCM8 was not detectable on chromatin, while MCM3 was bound. In S phase (60 minutes) MCM8 was bound to chromatin and showed a fine punctuate staining pattern different from that of MCM3, which was more homogenous. In late S phase (90 minutes), MCM3 was released from chromatin while MCM8 remained bound (FIG. 3A), confirming data shown in FIG. 2.

When pre-RCs formation was inhibited by blocking MCM2-7 loading with geminin, both MCM8 and MCM3 were not chromatin-bound (FIG. 3A, Gem). However, in the presence of the S-CDK inhibitor p21, which interferes with pre-ICs formation, but not pre-RCs (Mimura and Takisawa, 1998), MCM3 bound to chromatin but MCM8 did not (FIG. 3B; p21^{I}). Therefore MCM8 loading requires both MCM2-7 and S-CDK activity. Consistent with this hypothesis, adding p21 after formation of pre-ICs, that is after initiation, did not block DNA synthesis as expected (Jackson et al., 1995 and data not shown) and MCM8 was chromatin-bound (FIG. 3B, p₂₁^{E}). These results indicate that formation of both pre-RCs and pre-ICs are required to load MCM8 onto chromatin and are consistent with MCM8 functioning at the onset of S-phase.

The Inventors further analyzed whether MCM8 binding to chromatin depends upon initiation or elongation steps of DNA synthesis using the DNA polymerases inhibitor aphidicolin. Addition of aphidicolin before initiation of DNA synthesis does not interfere with formation of both pre-ICs and initiation complexes. However, formation of the elongation complex is blocked causing a strong inhibition of DNA synthesis (less than 1%, data not shown). In these conditions the elongation factor PCNA which requires DNA synthesis to bind (Michael et al., 2000; Mimura et al., 2000; Waga, 1994) did not associate with chromatin (FIG. 3C, lanes 3-4). MCM8 is barely detectable on chromatin in the presence of aphidicolin, while MCM3 is bound as expected (FIG. 3B, aphi^{I} and panel C, lanes 3-4). DNA polymerase α accumulates on chromatin (FIG. 3C, lanes 3-4), very likely due to extensive DNA unwinding (Michael et al., 2000; Walter and Newport, 2000) which is dependent upon the activity of MCM2-7 proteins (Pacek and Walter, 2004; Shechter et al., 2004). In contrast, when aphidicolin is added during elongation, DNA replication quickly arrests (data not shown) but MCM8 remains chromatin-bound (FIG. 3B, aphi^{E} and panel D, lane 2). Accumulation of DNA polymerase α is observed as expected, (FIG. 3D, lane 2) while MCM8 remains bound and does not accumulate (lane 2). This result indicates that in contrast to MCM2-7 (see FIG. 3B and Chong et al., 1995; Coué et al., 1996), loading of MCM8 onto chromatin requires the elongation step of DNA synthesis.

### Example 4

### MCM8 is Required for Processive DNA Synthesis

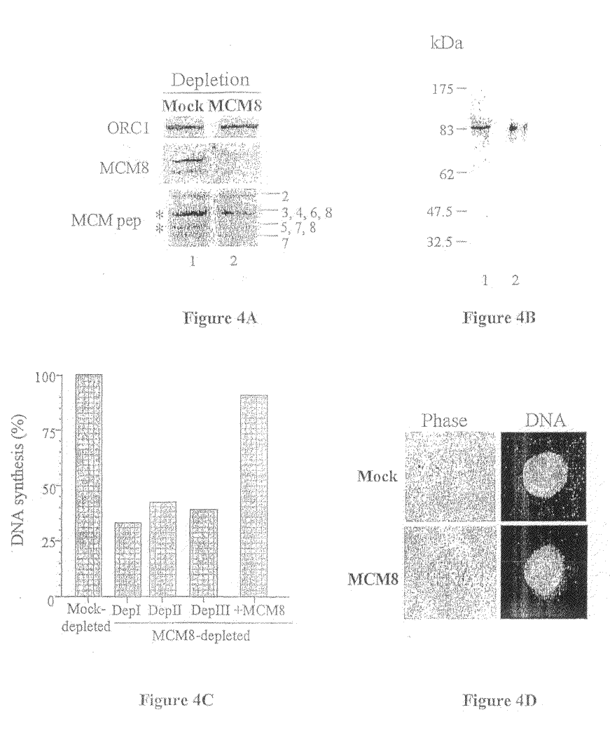

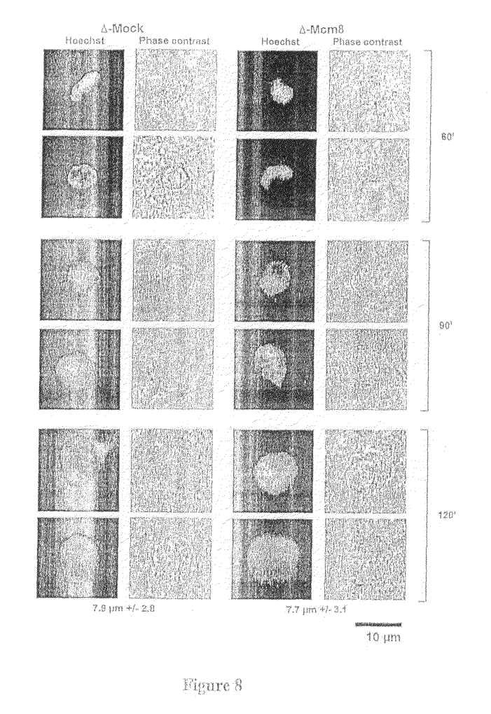

Xenopus extracts were immunodepleted of MCM8 and DNA synthesis was compared to control extracts depleted with non-specific antibodies. Depletion of MCM8 (FIG. 4A) did not remove ORC1, nor MCM2-7 proteins from extracts, confirming that MCM8 is not associated with these proteins. However chromosomal DNA replication was inhibited to around 40% of the control (FIG. 4C). This defect was recovered by addition of MCM8 purified from egg cytoplasm (FIGS. 4B and 4C, +MCM8). The Inventors also confirmed that nuclei formed normally in absence of MCM8 (FIG. 4D, phase and FIG. 8).

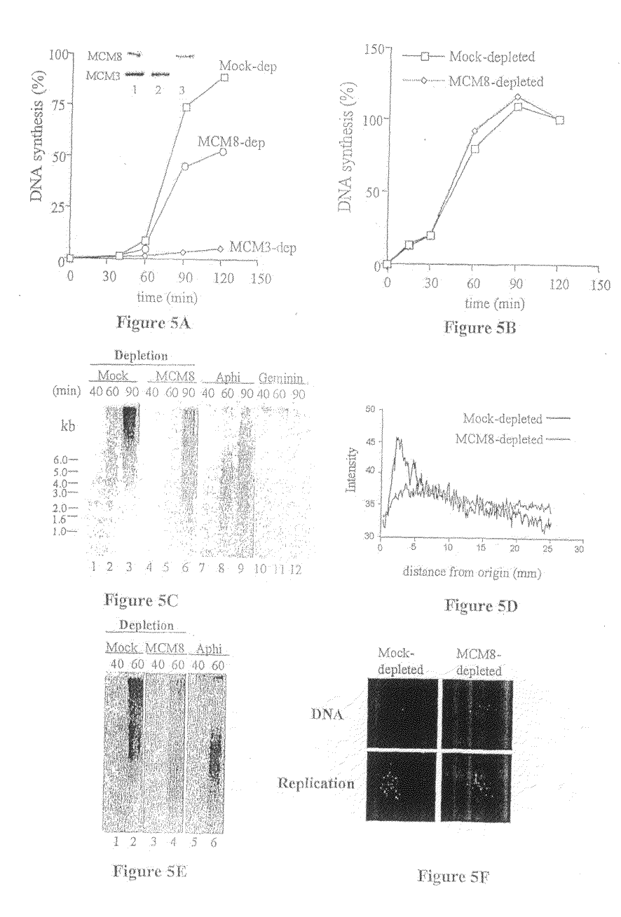

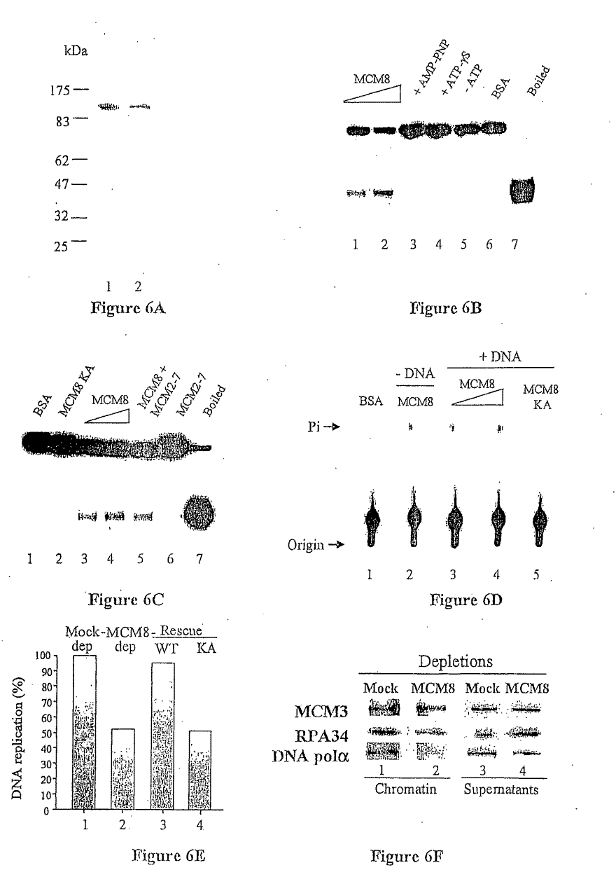

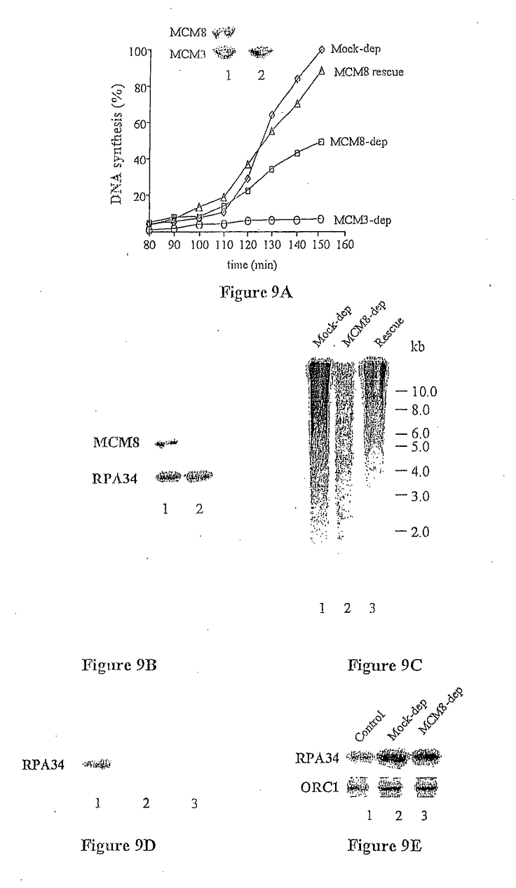

The phenotype of MCM8-depleted extracts is rather different from that observed by removal of a single MCM2-7 protein, which results in complete inhibition of DNA synthesis (Hennessy et al., 1991; Kubota et al., 1997; Labib et al., 2000; Liang et al., 1999; Madine et al., 1995a; Maiorano et al., 1996; Maiorano et al., 2000a). Thus, removal of MCM2-7 proteins with an MCM3 antibody (FIG. 5A, diamonds), completely abolished replication, as expected (Chong et al., 1995; Kubota et al., 1995; Madine et al., 1995a; Maiorano et al., 2000a). However, upon removal of MCM8 (FIG. 5A, circles), DNA replication initiated at the same time as in mock-depleted extracts (FIG. 5A, squares), but the rate of chromosomal DNA synthesis is slower. Even when more than 99% of the MCM8 protein was removed from extracts, the Inventors still observed a slow replication phenotype, which was efficiently rescued by recombinant MCM8 (FIG. 9A and FIG. 6E). Complementary DNA synthesis on a single-stranded DNA was not affected by removal of MCM8 (FIG. 5B. On this substrate DNA synthesis is strictly dependent on DNA primase/polymerase a activity (Mechali and Harland, 1982) but replication forks are not built and DNA unwinding is not required. In contrast other events occurring at the replication fork are executed, including RNA priming by DNA primase/polymerase α, DNA chain elongation and nucleosome assembly coupled to DNA synthesis (Almouzni and Mechali, 1988; Mechali and Harland, 1982). This result suggests that MCM8 is not directly required for the enzymatic activity of DNA polymerase α.

To further investigate the phenotype of MCM8-depleted extracts, DNA replication products were analyzed by alkaline gel electrophoresis (FIG. 5C). Replicating DNA is expected to migrate as a smear accumulating high molecular weight DNA chains while partially replicated or slowly replicating DNA should give an extended smear corresponding to accumulation of replication intermediates (nascent DNA). FIGS. 5C-D show that DNA chains synthesized in MCM8-depleted extracts are shorter than those observed in control extracts (lanes 1-6). Replication intermediates observed in MCM8-depleted egg extracts could be reversed to high molecular weight DNA chains by addition of recombinant MCM8 (FIG. 9C), demonstrating that the phenotype is specific to MCM8. The size of DNA synthesized in MCM8-depleted extracts was comparable to that obtained by slowing down replication forks with a low concentration of aphidicolin (lanes 7-9 and FIG. 5E). The phenotype obtained by MCM8 depletion also differs from that obtained by blocking replication at initiation with geminin, in which no nascent DNA is detected (FIG. 5C, lanes 10-12), or by depletion of MCM2-7 proteins (Françon et al., 2004).

Analysis of DNA synthesis in situ, was addressed by incorporation of the nucleotide analogue biotin-dUTP (FIG. 5F). Nuclei assembled in mock-depleted extracts accumulated a homogenous staining whereas nuclei assembled in MCM8-depleted extracts showed a punctuate pattern of biotin incorporation (Replication), reminiscent of that obtained by slowing down DNA synthesis with aphidicolin (Dimitrova and Gilbert, 2000). From these results altogether, the Inventors suggest that MCM8 is not required for initiation but functions during processive DNA synthesis, regulating the rate of replication forks movement.

### Example 5

### MCM8 Displays DNA Helicase and DNA-dependent ATPase Activity in Vitro

MCM8 contains ATP binding and hydrolysis motifs hinting to a function in unwinding as helicase, that would be consistent with phenotypes observed in MCM8-depleted extracts. Recombinant wild-type as well as a mutant in the ATP binding site (Walker A motif) were made and purified from insect cells (FIG. 6A). Significant DNA helicase activity, as determined by displacement of a 40 bases labelled oligonucleotide annealed to single stranded DNA, was detected with recombinant MCM8 (FIG. 6B, lanes 1-2). No such activity could be detected in the presence of non hydrolyzable ATP substrates (lane 3-4), nor in the absence of ATP (lane 5). Accordingly, the Inventors did not detect any helicase activity with MCM8 mutated in the ATP binding site (MCM8 K to A⁴⁵⁵, FIG. 6C, lane 2), nor with the MCM2-7 complex (lane 6) as expected, since the purified heterohexamer is inactive (Lee and Hurwitz, 2000). Significantly, the MCM2-7 complex did not stimulate the MCM8 helicase activity (lane 5) compared to wild-type MCM8 (lanes 3-4). ATP hydrolysis is detected with recombinant MCM8, which is stimulated by DNA (FIG. 6D, lane 2-4). Only background ATP hydrolysis was observed with MCM8 bearing the mutated ATP binding site (lane 5). This result suggests that ATP hydrolysis catalyzed by MCM8 requires an intact Walker A motif. Finally, this mutant did not rescue DNA replication in MCM8-depleted extracts, while replication was efficiently rescued by wild-type MCM8 (FIG. 6E). This result suggests that ATP hydrolysis catalyzed by MCM8 is required to efficiently replicate chromosomal DNA.

The Inventors conclude that MCM8 displays both DNA helicase and DNA-dependent ATPase activity in vitro in a reaction that does not require the MCM2-7 complex.

### Example 6

### MCM8 Regulates Efficient Assembly of RPA34 and DNA Polymerase α Onto Replicating Chromatin

A main function of the helicase during S-phase is to unwind DNA, leading to production of single-stranded DNA. This substrate is recognized by the trimeric RPA complex in concerted action with DNA polymerase α at replication forks (Waga, 1994). The Inventors wished to analyze whether MCM8 may be implicated in this reaction. In the absence of MCM8 the chromatin binding of MCM3 was not affected (FIG. 6F, lane 2), consistent with MCM8 binding to chromatin after MCM2-7 (FIG. 2A and FIG. 3). This result also demonstrates that MCM8 is not required for MCM2-7 chromatin loading. However, the amount of chromatin-bound DNA polymerase α, and to a lesser extent that of RPA34, was reduced (FIG. 6F, compare lanes 1 and 2), while the binding of Cdc45, required to recruit DNA polymerase α (Mimura and Takisawa, 1998), was not significantly affected. Failure of both RPA34 and DNA polymerase α to accumulate on chromatin was not due to co-depletion (FIG. 6F, lanes 3-4). In addition, neither RPA34 (FIG. 9D) nor DNA polymerase α (data not shown), were detected in MCM8 immunoprecipitates, further suggesting that these proteins do not form a complex in egg extracts. Finally, following a replication block at initiation, RPA34 accumulated onto Chromatin in MCM8-depleted extracts as in mock-depleted extracts (FIG. 9E). This result suggests that MCM8 is dispensable for unwinding at initiation, a reaction which is mainly catalyzed by MCM2-7 proteins (Pacek and Walter, 2004; Shechter et al., 2004), and is consistent with the observation that MCM8 is not chromatin-bound in these conditions (FIG. 3C). The Inventors conclude that MCM8 is required for accumulation and/or retention of RPA34 and DNA polymerase α on replicating chromatin.

### Example 7

### MCM8 Co-localizes With Replication Foci and RPA34 on Chromatin Once DNA Synthesis is Initiated

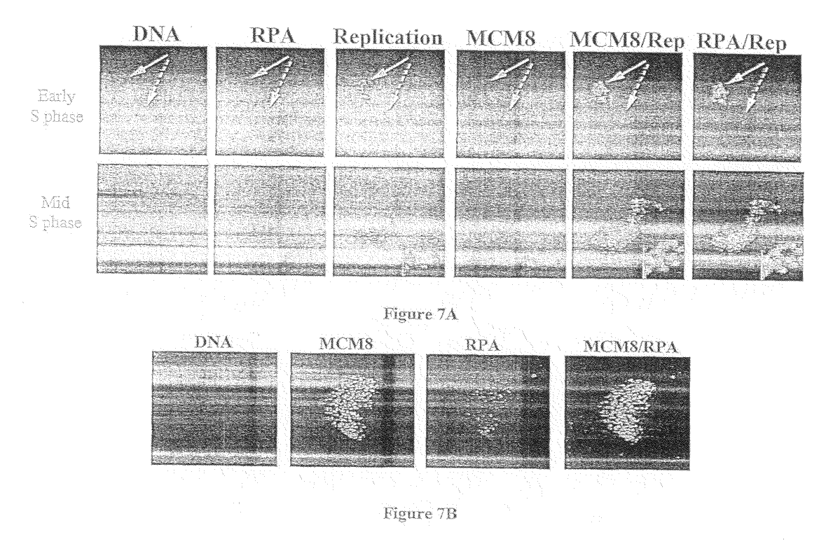

The Inventors analyzed the distribution of both MCM8 and RPA34 proteins on replicating chromatin. Nuclei formed in Xenopus egg extracts were pulse-labelled with the nucleotide analogue biotin-dUTP, in early S phase, when RPA foci appear on chromatin. In Xenopus RPA forms foci on chromatin before initiation of DNA synthesis, and after initiation, at replication forks (Adachi and Laemmli, 1992; Françon et al., 2004). As expected, RPA foci are detected both on regions already replicating (FIG. 7A, biotin-dUTP positive, arrow) and on regions not yet engaged in DNA synthesis (biotine-dUTP negatives, dashed arrow). In contrast, MCM8 was exclusively associated with replicating chromatin which stained positive for RPA. In mid to late S phase, all RPA foci co-localized with biotin-dUTP foci that also contained MCM8 (FIG. 7A, insets). The distribution of MCM8 on chromatin was rather different from that of MCM3, whose diffuse staining did not co-localize with replication foci (Nadine et al., 1995b), similar to MCM4 (Coué et al., 1996) and data not shown). To further confirm that MCM8 associates with RPA once DNA synthesis is initiated, nuclei were formed in egg extracts in presence of aphidicolin at low concentration. This treatment allows DNA replication initiation but slows down the elongation process and stabilizes RPA foci. In these conditions, MCM8 is chromatin-bound and an extensive co-localization (over 90%) of MCM8 and RPA foci was observed (FIG. 7B). The Inventors conclude that MCM8 assembles at replication foci only when DNA synthesis is initiated, at structures containing RPA.

### Conclusion

The function of MCM8 appears to be distinct from that of MCM2-7 in several aspects. First, MCM8 associates with chromatin only after licensing has occurred (that is after loading of MCM2-7), at the onset of DNA synthesis. Its association with chromatin coincides with the release of the licensing factor Cdt1, suggesting that Cdt1 is not directly required for MCM8 chromatin loading. This conclusion is also supported by the observation that removal of Cdt1 from chromatin after licensing, but before initiation, does not affect the rate of DNA synthesis (Maiorano et al., 2004) and see below). Given that this treatment also removes ORC1 and Cdc6 from chromatin (Rowles et al., 1999), it suggests that these proteins are neither directly required for the chromatin assembly of MCM8. Consistent with this conclusion, the Inventors have shown that MCM8 does not bind to chromatin in membrane-depleted egg extracts which assemble pre-RCs but cannot initiate DNA synthesis.

Second, the recruitment of MCM8 on chromatin requires that DNA synthesis is initiated. In contrast, MCM2-7 proteins accumulate on chromatin before and independently of DNA polymerases function (Chong et al., 1995; Coué et al., 1996), consistent with their role in forming pre-RCs. Third, MCM8 does not form complexes with MCM2-7 proteins in egg extracts and does not co-localize with MCM3 on chromatin (data not shown). Fourth, MCM3 accumulates normally on chromatin in the absence of MCM8, indicating that MCM8 is not required for licensing. Furthermore, in the absence of MCM8 the rate of DNA synthesis is slowed down and nascent DNA accumulates, while no replication is observed by removal of MCM2-7 proteins. Finally, MCM8 accumulates on chromatin upon initiation of DNA synthesis while MCM2-7 proteins are removed by replication forks progression. Overall, these results indicate that MCM8 is not implicated in initiation of DNA synthesis, as for the MCM2-7 proteins.

The phenotype of MCM8-depleted egg extracts, and the dynamics of MCM8 chromatin binding, suggest a specific role for MCM8 during processive DNA synthesis. In the absence of MCM8 the rate of DNA synthesis is decreased. DNA helicase and DNA-dependent ATPase activity are associated with recombinant MCM8 in vitro, and both activities are abolished by mutating the ATP binding site of MCM8. This mutant does not rescue DNA replication in MCM8-depleted egg extracts. The finding that recombinant MCM8 displays ATPase and DNA helicase activity in vitro by itself is rather unique as no helicase nor ATPase activity has been so far reported for a single eukaryotic MCM2-7 protein, but only for a sub-set of these proteins (Lee and Hurwitz, 2000; You et al., 1999). Moreover, in the absence of MCM8 the recruitment of RPA34 and DNA polymerase α is reduced suggesting that MCM8 regulates the association of these proteins with replicating chromatin. From these observations altogether, the Inventors propose that MCM8 functions during DNA synthesis in unwinding as a DNA helicase. The low levels of RPA34 and DNA polymerase a observed in the absence of MCM8 could be explained as reduction of DNA unwinding during elongation. Single-stranded DNA (ssDNA), generated by unwinding, is recognized and bound by RPA which is essential for loading DNA polymerase α at replication forks (Mimura and Takisawa, 1998; Walter and Newport, 2000).

Unwinding can be uncoupled from DNA polymerase activity, so that inhibiting DNA polymerases does not result in inhibition of the helicase on a few kilobase pairs (Michael et al., 2000; Walter and Newport, 2000). Accordingly to this model, the Inventors observed that MCM8 remains chromatin-bound by blocking DNA synthesis with aphidicolin during elongation, while DNA polymerase α accumulates as a result of binding to ssDNA generated by the helicase. In contrast, when DNA synthesis is inhibited with aphidicolin at initiation, MCM8 does not bind to chromatin and unwinding occurs normally due to the activity of MCM2-7 proteins (Pacek and Walter, 2004; Shechter et al., 2004) which remain chromatin-bound at this stage. It cannot be excluded that MCM8 might also participate in the replication of specialized portions of the genome (e.g., heterochromatin), during the termination of DNA synthesis or in other aspects of DNA metabolism, such as DNA repair or recombination. The features of MCM8 are compatible with these possibilities.

MCM2-7 proteins do not co-localize with replication foci and RPA (Coué et al., 1996; Dimitrova et al., 1999; Laskey and Madine, 2003) leading to a paradox in the understanding of DNA synthesis in eukaryotes; if MCM2-7 proteins are the replicative helicase, why then no interaction with the DNA synthesis machinery is observed? The distribution of MCM8 on chromatin coincides with that of DNA replication foci and RPA34, providing one explanation to this paradox in vertebrates, as MCM8 links licensing to processive DNA synthesis at replication factories. The results presented here are consistent with a model in which MCM2-7 proteins induce the first unwinding at DNA replication origins to allow assembly of the replisome and recruitment of MCM8 onto chromatin. This conclusion is consistent with the observation that not only both pre-RC and pre-IC are required for MCM8 chromatin binding, but also that DNA synthesis must have initiated. MCM8 contributes to unwinding as DNA helicase during the progression of replication forks, by itself or in association with MCM2-7 proteins, or might perhaps replace some subunits within the whole MCM2-7 complex. Although the Inventors have not seen any stimulation of MCM8 helicase activity by the MCM2-7 complex in vitro, this latter possibility cannot completely ruled out. In both cases, however, MCM8 is present at replication foci where it is involved in replication fork progression.

Based on sequence comparison, a homolog of MCM8 is not found in the genome of yeast and worms (Gozuacik et al., 2003 and data not shown). The requirement for MCM8 might be related to the size and/or the complexity of the genome, so that the presence of an additional helicase factor may be required to ensure efficient processivity in replicating large genomes. Another possibility would be that in simple eukaryotes another helicase, not yet identified, but unrelated to MCM8, fulfils a similar function.

### REFERENCES

Adachi, Y., and Laemmli, U. K. (1992). Identification of nuclear pre-replication centers poised for DNA synthesis in Xenopus egg extracts: immunolocalization study of replication protein A. J Cell Biol 119, 1-15.
Adachi, Y., and Yanagida, M. (1989). Higher order chromosome structure is affected by cold-sensitive mutations in a Schizosaccharomyces pombe gene crm1+ which encodes a 115-kD protein preferentially localized in the nucleus and its periphery. J Cell Biol 108, 1195-1207.
Almouzni, G., and Mechali, M. (1988). Assembly of spaced chromatin promoted by DNA synthesis in extracts from Xenopus eggs. Embo J 7, 665-672.
Aparicio, O. M., Weinstein, D. M., and Bell, S. P. (1997). Components and dynamics of DNA replication complexes in S. cerevisiae: redistribution of MCM proteins and Cdc45p during S phase. Cell 91, 59-69.
Bell, S. P., and Dutta, A. (2002). DNA replication in eukaryotic cells. Annu Rev Biochem 71, 333-374.
Blow, J. J., and Laskey, R. A. (1986). Initiation of DNA replication in nuclei and purified DNA by a cell-free extract of Xenopus eggs. Cell 47, 577-587.
Chong, J. P., Mahbubani, H. M., Khoo, C.-Y., and Blow, J. J. (1995). Purification of an MCM-containing complex as a component of the DNA replication licensing system. Nature 375, 418-421.
Coleman, T. R., Carpenter, P. B., and Dunphy, G. (1996). The Xenopus Cdc6 protein is essential for the initiation of a single round of DNA replication in cell-free extracts. Cell 87, 53-63.
Coue, M., Amarglio, F., Maiorano, D., Bocquet, S., and Mechali, M. (1998). Evidence for different MCM subcomplexes with differential binding to chromatin in Xenopus. Exp Cell Res 245, 282-289.
Coué, M., Kearsey, S. E., and Mechali, M. (1996). Chromatin binding, nuclear localization and phosphorylation of Xenopus cdc21 are cell-cycle dependent and associated with the control of initiation of DNA replication. EMBO J 15, 1085-1097.
Davey, M. J., Indiani, C., and O'Donnell, M. (2003). Reconstitution of the Mcm2-7p heterohexamer, subunit arrangement, and ATP site architecture. J Biol Chem 278, 4491-4499.
Dimitrova, D. S., and Gilbert, D. M. (2000). Temporally coordinated assembly and disassembly of replication factories in the absence of DNA synthesis. Nat Cell Biol 2, 686-694.
Dimitrova, D. S., Todorov, I. T., Melendy, T., and Gilbert, D. M. (1999). Mcm2, but not RPA, is a component of the mammalian early GI-phase prereplication complex. J Cell Biol 146, 709-722.
Dorneiter, I., Erdile, L. F., Gilbert, I. U., von Winker, D., kelly, T. J. and Fanning, E. (1992). Interaction of DNA polymerase a-primase with cellular replication protein A and SV40 T antigen. EMBO 11, 769-776.
Françon, P., Lemaitre, J.-M., Dryer, C., Maiorano, D., Cuvier, O., and Méchali, M. (2004). A hypophosphorylated form of RPA 34 is a specific component of pre-replication centers. J Cell Science in press.
Gozuacik, D., Chami, M., Lagorce, D., Faivre, J., Murakami, Y., Poch, O., Biermann, E., Knippers, R., Brechot, C., and Paterlini-Brechot, P. (2003). Identification and functional characterization of a new member of the human Mcm protein family: hMcm8. Nucleic Acids Res 31, 570-579.
Gozuacik, D., Murakami, Y., Saigo, K., Chami, M., Mugnier, C., Lagorce, D., Okanoue, T., Urashima, T., Brechot, C., and Paterlini-Brechot, P. (2001). Identification of human cancer-related genes by naturally occurring Hepatitis B Virus DNA tagging. Oncogene 20, 6233-6240.
Hennessy, K. M., Lee, A., Chen, E., and Botstein, D. (1991). A group of interacting yeast DNA replication genes. Genes & Dev 5, 958-969.
Ishimi, Y. (1997). A DNA helicase activity is associated with an MCM4, -6, and -7 protein complex. J Biol Chem 272, 24508-24513.
Ishimi, Y., Komamura, Y., You, Z., and Kimura, H. (1998). Biochemical function of mouse minichromosome maintenance 2 protein. J Biol Chem 273, 8369-8375.
Jackson, P. K., Chevalier, S., Philippe, M., and Kirschner, M. W. (1995). Early events in DNA replication require cyclin E and are blocked by p21CIP1. J Cell Biol 130, 755-769.
Johnson, E. M., Kinoshita, Y., and Daniel, D. C. (2003). A new member of the MCM protein family encoded by the human MCM8 gene, located contrapodal to GCD 10 at chromosome band 20p12.3-13. Nucleic Acids Res 31, 2915-2925.
Kearsey, S. E., and Labib, K. (1998). MCM proteins: evolution, properties, and role in DNA replication. Biochim Biophys Acta 1398, 113-136.
Krude, T., Musahl, C., Laskey, R. A., and Knippers, R. (1996). Human replication proteins hCdc21, hCdc46 and P1Mcm3 bind chromatin uniformly before S-phase and are displaced locally during DNA replication. J Cell Sci 109, 309-318.
Kubota, Y., Mimura, S., Nishimoto, S.-I., Masuda, T., Nojima, H., and Takisawa, H. (1997). Licensing of DNA replication by a multi-protein complex of MCM/P1 proteins in Xenopus eggs. EMBO J 16, 3320-3331.
Kubota, Y., Mimura, S., Nishimoto, S.-I., Takisawa, H., and Nojima, H. (1995). Identification of the yeast MCM3-related protein as a component of Xenopus DNA replication licensing factor. Cell 81, 601-609.
Labib, K., and Diffley, J. F. (2001). Is the MCM2-7 complex the eukaryotic DNA replication fork helicase? Curr Opin Genet Dev 11, 64-70.
Labib, K., Diffley, J. F., and Kearsey, S. E. (1999). G1-phase and B-type cyclins exclude the DNA-replication factor Mcm4 from the nucleus. Nat Cell Biol 1, 415-422.
Labib, K., Tercero, J. A., and Diffley, J. F. (2000). Uninterrupted MCM2-7 function required for DNA replication fork progression. Science 288, 1643-1647.
Laskey, R. A., and Madine, M. A. (2003). A rotary pumping model for helicase function of MCM proteins at a distance from replication forks. EMBO Rep 4, 26-30.
Lee, J. K., and Hurwitz, J. (2000). Isolation and characterization of various complexes of the minichromosome maintenance proteins of Schizosaccharomyces pombe. J Biol Chem 275, 18871-18878.
Lee, J. K., and Hurwitz, J. (2001). Processive DNA helicase activity of the minichromosome maintenance proteins 4, 6, and 7 complex requires forked DNA structures. Proc Natl Acad Sci USA 98, 54-59.
Leibovici, M., Monod, G., Geraudie, J., Bravo, R, and Mechali, M. (1992). Nuclear distribution of PCNA during embryonic development in Xenopus laevis: a reinvestigation of early cell cycles. J Cell Sci 102 (Pt 1), 63-69.
Liang, D. T., Hodson, J. A., and Forsburg, S. L. (1999). Reduced dosage of a single fission yeast MCM protein causes genetic instability and S phase delay. J Cell Sci 112 (Pt 4), 559-567.
Madine, M. A., Khoo, C.-Y., Mills, A. D., and Laskey, R. A. (1995a). MCM3 complex required for cell cycle regulation of DNA replication in vertebrate cells. Nature 375, 421-424. Madine, M. A., Khoo, C.-Y., Mills, A. D., Musahl, C., and LAskey, R. (1995b). The nuclear envelope prevents reinitiation of replication by regulating the binding of MCM3 to chromatin in Xenopus egg extracts. Curr Biol 5, 1270-1279.
Maiorano, D., Blom van Assendelft, G., and Kearsey, S. E. (1996). Fission yeast cdc21, a member of the MCM protein family, is required for onset of S phase and is located in the nucleus throughout the cell cycle. EMBO J 15, 861-872.
Maiorano, D., Lemaitre, J. M., and Mechali, M. (2000a). Stepwise regulated chromatin assembly of MCM2-7 proteins. J Biol Chem 275, 8426-8431.
Maiorano, D., Moreau, J., and Mechali, M. (2000b). XCDT1 is required for the assembly of pre-replicative complexes in Xenopus laevis. Nature 404, 622-625.
Maiorano, D., Rul, W., and Mechali, M. (2004). Cell cycle regulation of the licensing activity of Cdt1 in Xenopus laevis. Exp Cell Res 295, 138-149.
Maiorano D, Cuvier O, Danis E, Mechali M. (2005). MCM8 is an MCM2-7-related protein that functions as a DNA helicase during replication elongation and not initiation. Cell, 120, 315-28.
McGarry, T. J., and Kirschner, M. W. (1998). Geminin, an inhibitor of DNA replication, is degraded during mitosis. Cell 93, 1043-1053.
McGlynn P, Lloyd R G. (2002). Recombinational repair and restart of damaged replication forks. Nat Rev Mol Cell Biol. 3, 859-70.
Mechali, M., and Harland, R. M. (1982). DNA synthesis in a cell-free system from Xenopus eggs: priming and elongation on single-stranded DNA in vitro. Cell 30, 93-101.
Melendy, T., and Stillman, B. (1993). An interaction between replication protein A and SV40 T antigen appears essential for primosome assembly during SV40 DNA replication. J Biol Chem 268, 3389-3395.
Mendez, J., and Stillman, B. (2003). Perpetuating the double helix: molecular machines at eukaryotic DNA replication origins. Bioessays 25, 1158-1167.
Menut, S., Lemaitre, J. M., Hair, A., and Mechali, M. (1988). DNA replication and chromatin assembly using Xenopus egg extracts. In Advances in Molecular Biology: A comparative Methods Approach to the Study of Ooocytes and Embryos, Oxford University Press, Ed J D Richter.
Michael, W. M., Ott, R., Fanning, E., and Newport, J. (2000). Activation of the DNA replication checkpoint through RNA synthesis by primase. Science 289, 2133-2137.
Mimura, S., Masuda, T., Matsui, T., and Takisawa, H. (2000). Central role for cdc45 in establishing an initiation complex of DNA replication in Xenopus egg extracts. Genes Cells 5, 439-452.
Mimura, S., and Takisawa, H. (1998). Xenopus Cdc45-dependent loading of DNA polymerase alpha onto chromatin under the control of S-phase Cdk. EMBO J 17, 5699-5707.
Pacek, M., and Walter, J. C. (2004). A requirement for MCM7 and Cdc45 in chromosome unwinding during eukaryotic DNA replication. Embo J 23, 3667-3676.
Rebagliati, M. R., Weeks, D. L., Harvey, R P., and Melton, D. A. (1985). Identification and cloning of localized maternal RNAs from Xenopus eggs. Cell 42, 769-777.
Romanowski, P., Madine, M. A., and Laskey, R. (1996). XMCM7, a novel member of the Xenopus MCM family, interacts with XMCM3 and colocalizes with it throughout replication. Proc Natl Acad Sci USA 93, 10189-10194.
Rowles, A., Tada, S., and Blow, J. J. (1999). Changes in association of the Xenopus origin recognition complex with chromatin on licensing of replication origins. J Cell Sci 112, 2011-2018.
Schwacha, A., and Bell, S. P. (2001). Interactions between two catalytically distinct MCM subgroups are essential for coordinated ATP hydrolysis and DNA replication. Mol Cell 8, 1093-1104.
Shechter, D., Ying, C. Y., and Gautier, J. (2004). DNA unwinding is an Mcm complex-dependent and ATP hydrolysis-dependent process. J Biol Chem 279, 45586-45593.
Tada, S., Li, A., Maiorano, D., Mechali, M., and Blow, J. J. (2001). Repression of origin assembly in metaphase depends on inhibition of RLF-B/Cdt1by geminin. Nat Cell Biol 3, 107-113.
Tanaka, T., Knapp, D., and Nasmyth, I C (1997). Loading of an MCM protein onto DNA replication origins is regulated by Cdc6p and CDKs. Cell 90, 649-660.
Todorov, I., Attaran, A., and Kearsey, S. E. (1995). BM28, a human member of the MCM2-3-5 family, is displaced from chromatin during DNA replication. J Cell Biol 129,1433-1445.
Tye, B. K. (1999). MCM proteins in DNA replication. Annu Rev Biochem 68, 649-686.
Vuillard, L., Rabilloud, T., and Goldberg, M. B. (1998). Interactions of non-detergent sulfobetaines with early folding intermediates facilitate in vitro protein renaturation. Eur J Biochem 256, 128-135.
Waga, S., and Stillman, B. (1994). Anatomy of a DNA replication fork revealed by reconstitution of SV40 DNA replication in vitro. Nature 369, 207-212.
Walter, J., and Newport, J. (2000). Initiation of eukaryotic DNA replication: origin unwinding and sequential chromatin association of Cdc45, RPA, and DNA polymerase alpha. Mol Cell 5, 617-627.
Wohlschlegel, J. A., Dwyer, B. T., Dhar, S. K., Cvetic, C., Walter, J. C., and Dutta, A. (2000). Inhibition of eukaryotic DNA replication by geminin binding to cdt1. Science 290, 2309-2312.
You, Z., Komamura, Y., and Ishimi, Y. (1999). Biochemical analysis of the intrinsic Mcm4-Mcm6-mcm7 DNA helicase activity. Mol Cell Biol 19, 8003-8015.

1-26. (canceled)

27. A method for the treatment of a human or animal pathology linked to a dysfunction of the expression of the MCM8 gene, or of human or animal cancers by administering to said human or animal the human or animal MCM8 gene coding for a DNA helicase, or parts of said gene, or transcripts thereof, or antisense nucleic acids able to hybridize with part of said transcripts, or silencing RNA derived from parts of said transcripts and able to repress said MCM8 gene, or proteins or peptidic fragments translated from said transcripts, or antibodies directed against said proteins or peptidic fragments.

28. The method according to claim 27, for the treatment of cancers, wherein the helicase activity of MCM8 in tumoral cells of the human or animal body is inactivated by using silencing iRNA according to RNA interference, selected from the group consisting of double-stranded RNA (dsRNA) for post-transcriptional gene silencing, short interfering RNA (siRNA) and short hairpin RNA (shRNA) to induce specific gene suppression, and antisense DNA or RNA, or antibodies, in order to curb the proliferation of said tumoral cells.

29. The method use according to claim 27, for the treatment of neoplastic diseases selected from the group consisting of choriocarcinoma, liver cancer induced by DNA damaging agents or by infection by Hepatitis B virus, skin melanotic melanoma, melanoma, premalignant actinic keratose, colon adenocarcinoma, basal cell carcinoma, squamous cell carcinoma, ocular cancer, non-Hodgkin's lymphoma, acute lymphocytic leukaemia, meningioma, soft tissue sarcoma, osteosarcoma, and muscle rhabdomyosarcoma or of brain diseases selected from the group consisting of Alzheimer disease, neuron degenerative diseases and mental retardation or of hematological disorders.

30. The method according to claim 27, for the treatment of a human or animal pathology linked to a dysfunction of the expression of the MCM8 gene, wherein the number of functional MCM8 helicases is increased or the activity of MCM8 helicases in cells of the human or animal body is stimulated by administration of functional MCM8 proteins or of fragments thereof or by gene or cell therapy.

31. The method according to claim 27, for the treatment of pathologies corresponding to a predisposition towards cancer or premature aging and being caused by a defect of the helicase function.

32. The method according to claim 31 wherein the pathology is selected from the group consisting of Bloom's syndrome, Werner's syndrome, ataxia-telangectasia, xerodermia pigmentosum, Cockayne's syndrome and Rothmund-Thomson's syndrome.

33. The method according to claim 27, wherein the human or animal MCM8 genes are chosen among:
the xenopus MCM8 nucleotide sequence represented by SEQ ID NO: 1 encoding the xenopus helicase represented by SEQ ID NO 2,
the human MCM8 nucleotide sequence represented by SEQ ID NO: 3 encoding the human helicase represented by SEQ ID NO: 4,
the human MCM8 nucleotide sequence represented by SEQ ID NO: 5 encoding the human helicase represented by SEQ ID NO: 6,
the human MCM8 nucleotide sequence represented by SEQ ID NO: 7 encoding the human helicase represented by SEQ ID NO: 8,
the human MCM8 nucleotide sequence represented by SEQ ID NO: 9 encoding the human helicase represented by SEQ ID NO: 10,
the human MCM8 nucleotide sequence represented by SEQ ID NO: 11 encoding the human helicase represented by SEQ ID NO: 12,
the human MCM8 nucleotide sequence represented by SEQ ID NO: 13 encoding the human helicase represented by SEQ ID NO: 14,
the human MCM8 nucleotide sequence represented by SEQ ID NO: 15 encoding the human helicase represented by SEQ ID NO: 16,
the murine MCM8 nucleotide sequence represented by SEQ ID NO: 17 encoding the murine helicase represented by SEQ ID NO: 18,
the murine MCM8 nucleotide sequence represented by SEQ ID NO: 19 encoding the murine helicase represented by SEQ ID NO: 20,
the murine MCM8 nucleotide sequence represented by SEQ ID NO: 21 encoding the murine helicase represented by SEQ ID NO: 22.

34. The method according to claim 27, wherein said parts of the MCM8 nucleotide sequence contain approximately 3 to 240 nucleotides, and comprise a segment which is essential for the helicase function of MCM8 protein, said segment being selected from the group consisting of:
the nucleotide sequence represented by SEQ ID NO: 23 of the xenopus MCM8 gene represented by SEQ ID NO: 1,
the nucleotide sequence represented by SEQ ID NO: 25 of the xenopus MCM8 gene represented by SEQ ID NO: 1,
the nucleotide sequence represented by SEQ ID NO: 27 of the human MCM8 gene represented by SEQ ID NO: 9 or SEQ ID : 15,
the nucleotide sequence represented by SEQ ID NO: 29 of the human MCM8 gene represented by SEQ ID NO: SEQ ID NO: 9 or SEQ ID: 15,
the nucleotide sequence represented by SEQ ID NO: 31 of the human MCM8 gene represented by SEQ ID NO: 3 or SEQ ID NO: 13,
the nucleotide sequence represented by SEQ ID NO: 33 of the human MCM8 gene represented by SEQ ID NO: 3 or SEQ ID NO: 13,
the nucleotide sequence represented by SEQ ID NO: 35 of the murine MCM8 gene represented by SEQ ID NO: 17,
the nucleotide sequence represented by SEQ ID NO: 37 of the murine MCM8 gene, represented by SEQ ID NO: 17,
the nucleotide sequence represented by SEQ ID NO: 39 of the murine MCM8 gene, represented by SEQ ID NO: 19,
the nucleotide sequence represented by SEQ ID NO: 41 of the murine MCM8 gene, represented by SEQ ID NO: 19,
or wherein said peptidic fragments contain approximately 4 to 90 amino acids, and comprise a segment which is essential for the helicase function of MCM8 protein and which is selected from the group consisting of:
the amino-acid sequence represented by SEQ ID NO: 24 of the xenopus MCM8 protein represented by SEQ ID NO: 2,
the amino-acid sequence represented by SEQ ID NO: 26 of the xenopus MCM8 protein represented by SEQ ID NO: 2,
the amino-acid sequence represented by SEQ ID NO: 28 of the human MCM8 protein represented by SEQ ID NO: 10 or SEQ ID: 16,
the amino-acid sequence represented by SEQ ID NO: 30 of the human MCM8 protein represented by SEQ ID NO: 10 or SEQ ID: 16,
the amino-acid sequence represented by SEQ ID NO: 32 of the human MCM8 protein represented by SEQ ID NO: 4 or SEQ ID: 14,
the amino-acid sequence represented by SEQ ID NO: 34 of the human MCM8 protein represented by SEQ ID NO: 4 or SEQ ID: 14,
the amino-acid sequence represented by SEQ ID NO: 36 of the murine MCM8 protein represented by SEQ ID NO: 18,
the amino-acid sequence represented by SEQ ID NO: 38 of the murine MCM8 protein represented by SEQ ID NO: 18,
the amino-acid sequence represented by SEQ ID NO: 40 of the murine MCM8 protein represented by SEQ ID NO: 20,
the amino-acid sequence represented by SEQ ID NO: 42 of the murine MCM8 protein represented by SEQ ID NO: 20.

35. The method according to claim 27, wherein said MCM8 gene or said parts of the MCM8 nucleotide sequence or said transcripts or said proteins or peptidic fragments contain at least one mutation, by deletion and/or addition and/or substitution of one or more nucleotide or amino-acid.

36. The method according to claim 35, wherein said mutation is located on a site of phosphorylation by CDKs, said site being selected from the group consisting of:
nucleotides 253-258 of the xenopus MCM8 gene represented by SEQ ID NO: 1, encoding amino-acids 85-86 of the xenopus MCM8 protein represented by SEQ ID NO: 2,
nucleotides 820-825 of the xenopus MCM8 gene represented by SEQ ID NO: 1, encoding amino-acids 274-275 of the xenopus MCM8 protein represented by SEQ ID NO: 2,
nucleotides 1771-1776 of the xenopus MCM8 gene represented by SEQ ID NO: 1, encoding amino-acids 591-592 of the xenopus MCM8 protein represented by SEQ ID NO: 2,
nucleotides 2026-2031 of the xenopus MCM8 gene represented by SEQ ID NO: 1, encoding amino-acids 676-677 of the Xenopus MCM8 protein represented by SEQ ID NO: 2,
nucleotides 2098-2103 of the xenopus MCM8 gene represented by SEQ ID NO: 1, encoding amino-acids 700-701 of the Xenopus MCM8 protein represented by SEQ ID NO: 2,
nucleotides 154-159 of the human MCM8 gene represented by SEQ ID NO: 9 or SEQ ID NO: 15, encoding amino-acids 52-53 of the human MCM8 protein represented by SEQ ID NO: 10 or SEQ ID NO: 16,
nucleotides 181-186 of the human MCM8 gene represented by SEQ ID NO: 9 or SEQ ID NO: 15, encoding amino-acids 61-62 of the human MCM8 protein represented by SEQ ID NO: 10 or SEQ ID NO: 16,
nucleotides 268-273 of the human MCM8 gene represented by SEQ ID NO: 9 or SEQ ID NO: 15, encoding amino-acids 90-91 of the human MCM8 protein represented by SEQ ID NO: 10 or SEQ ID NO: 16,
nucleotides 838-843 of the human MCM8 gene represented by SEQ ID NO: 9 or SEQ ID NO: 15, encoding amino-acids 280-281 of the human MCM8 protein represented SEQ ID NO: 10 or SEQ ID NO: 16,
nucleotides 1786-1791 of the human MCM8 gene represented by SEQ ID NO: 9 or SEQ ID NO: 15, encoding amino-acids 596-597 of the human MCM8 protein represented SEQ ID NO: 10 or SEQ ID NO: 16,
nucleotides 2116-2121 of the human MCM8 gene represented by SEQ ID NO: 9 or SEQ ID NO: 15, encoding amino-acids 706-707 of the human MCM8 protein represented by SEQ ID NO: 10 or SEQ ID NO: 16,
nucleotides 1738-1743 of the human MCM8 gene represented by SEQ ID NO: 3 or SEQ ID NO: 13, encoding amino-acids 580-581 of the human MCM8 protein represented by SEQ ID NO: 4 or SEQ ID NO: 14,
nucleotides 2068-2073 of the human MCM8 gene represented by SEQ ID NO: 3 or SEQ ID NO: 13, encoding amino-acids 690-691 of the human MCM8 protein represented by SEQ ID NO: 4 or SEQ ID NO: 14,
nucleotides 247-252 of the murine MCM8 gene represented by SEQ ID NO: 17, encoding amino-acids 83-84 of the murine MCM8 protein represented by SEQ ID NO: 18,
nucleotides 817-822 of the murine MCM8 gene represented by SEQ ID NO: 17, encoding amino-acids 273-274 of the murine MCM8 protein represented by SEQ ID NO: 18,
nucleotides 1765-1770 of the murine MCM8 gene represented by SEQ ID NO: 17, encoding amino-acids 589-590 of the murine MCM8 protein represented by SEQ ID NO: 18,
nucleotides 163-168 of the murine MCM8 gene represented by SEQ ID NO: 19, encoding amino-acids 55-56 of the murine MCM8 protein represented by SEQ ID NO: 20,
nucleotides 733-738 of the murine MCM8 gene represented by SEQ ID NO: 19, encoding amino-acids 245-246 of the murine MCM8 protein represented by SEQ ID NO: 20,
nucleotides 1681-1686 of the murine MCM8 gene represented by SEQ ID NO: 19, encoding amino-acids 561-562 of the murine MCM8 protein represented by SEQ ID NO: 20,
and nucleotides 2011-2016 of the murine MCM8 gene represented by SEQ ID NO: 19, encoding amino-acids 671-672 of the murine MCM8 protein represented by SEQ ID NO: 20.

37. The method according to claim 35, wherein said mutations are chosen among the followings:
modification of the conserved threonine (T) in the TP motif to alanine (A) or an equivalent amino acid and modification of the conserved serine (S) in the SP motif to alanine (A) or an equivalent amino acid,
modification of the conserved threonine (T) in the TP motif to glutamate (E) or an equivalent amino acid and modification of the conserved serine (S) in the SP motif to glutamate (E) or an equivalent amino acid.

38. The method according to claim 35, wherein said mutation is located on a position which is essential for the helicase function of MCM8 protein, and is selected from the group consisting of:
the nucleotide sequence represented by SEQ ID NO: 23 of the xenopus MCM8 gene represented by SEQ ID NO: 1, encoding the amino-acid sequence represented by SEQ ID NO: 24 of the xenopus MCM8 protein represented by SEQ ID NO: 2,
the nucleotide sequence represented by SEQ ID NO: 25 of the xenopus MCM8 gene represented by SEQ ID NO: 1, encoding the amino-acid sequence represented by SEQ ID NO: 26 of the xenopus MCM8 protein represented by SEQ ID NO: 2,
the nucleotide sequence represented by SEQ ID NO: 27 of the human MCM8 gene represented by SEQ ID NO: 9 or SEQ ID NO: 15, encoding the amino-acid sequence represented by SEQ ID NO: 28 of the human MCM8 protein represented by SEQ ID NO: 10 or SEQ ID NO: 16,
the nucleotide sequence represented by SEQ ID NO: 29 of the human MCM8 gene represented by SEQ ID NO: 9 or SEQ ID NO: 15, encoding the amino-acid sequence represented by SEQ ID NO: 30 of the human MCM8 protein represented by SEQ ID NO: 10 or SEQ ID NO: 16,
the nucleotide sequence represented by SEQ ID NO: 31 of the human MCM8 gene represented by SEQ ID NO: 3 or SEQ ID NO: 13, encoding the amino-acid sequence represented by SEQ ID NO: 32 of the human MCM8 protein represented by SEQ ID NO: 4 or SEQ ID NO: 14,
the nucleotide sequence represented by SEQ ID NO: 33 of the human MCM8 gene represented by SEQ ID NO: 3 or SEQ ID NO: 13, encoding the amino-acid sequence represented by SEQ ID NO: 34 of the human MCM8 protein represented by SEQ ID NO: 4 or SEQ ID NO: 14,
the nucleotide sequence represented by SEQ ID NO: 35 of the murine MCM8 gene represented by SEQ ID NO: 17, encoding the amino-acid sequence represented by SEQ ID NO: 36 of the murine MCM8 protein represented by SEQ ID NO: 18,
the nucleotide sequence represented by SEQ ID NO: 37 of the murine MCM8 gene represented by SEQ ID NO: 17, encoding the amino-acid sequence represented by SEQ ID NO: 38 of the murine MCM8 protein represented by SEQ ID NO: 18,
the nucleotide sequence represented by SEQ ID NO: 39 of the murine MCM8 gene represented by SEQ ID NO: 19, encoding the amino-acid sequence represented by SEQ ID NO: 40 of the murine MCM8 protein represented by SEQ ID NO: 20,
the nucleotide sequence represented by SEQ ID NO: 41 of the murine MCM8 gene represented by SEQ ID NO: 19, encoding the amino-acid sequence represented by SEQ ID NO: 42 of the murine MCM8 protein represented by SEQ ID NO: 20.

39. The method according to claim 35, wherein said mutations are chosen among the followings:
modification of the conserved lysine (K) in the Walker A motif GxxGxGK to alanine (A) or threonine (T) or other non polar or polar neutral amino acids,
modification of the conserved aspartic acid (D) in the Walker B motif DExx to alanine (A) or threonine (T) or other non polar or polar neutral amino acids.

40. A method to induce the transformation of non tumoral cells into tumoral cells, by means of inhibitors of the MCM8 protein chosen among antisense nucleic acids, silencing RNA and antibodies directed against MCM8.

41. A method for the screening of biologically active agents useful in the treatment of human or animal pathology linked to a dysfunction of the expression of the MCM8 gene, said method comprising:
administering a potential agent to a non-human transgenic animal model for MCM8 gene function, selected from the group consisting of a MCM8 knock-out model and a model of exogenous and stably transmitted MCM8 sequence, and
determining the effect of said agent on the development of the transgenic animal and/or the development of diseases selected from the group comprising neoplastic diseases, selected from the group consisting of choriocarcinoma, liver cancer induced by DNA damaging agents or by infection by Hepatitis B virus, skin melanotic melanoma, melanoma, premalignant actinic keratose, colon adenocarcinoma, basal cell carcinoma, squamous cell carcinoma, ocular cancer, non-Hodgkin's lymphoma, acute lymphocytic leukaemia, meningioma, soft tissue sarcoma, osteosarcoma, and muscle rhabdomyosarcoma, brain diseases, selected from the group comprising Alzheimer disease, neuron degenerative diseases and mental retardation, hematological disorders and pathologies corresponding to a predisposition towards cancer or premature aging and being caused by a defect of the helicase function and being selected from the group comprising Bloom's syndrome, Werner's syndrome, ataxia-telangectasia, xerodermia pigmentosum, Cockayne's syndrome and Rothmund-Thomson's syndrome.

42. A method for the in vitro or ex vivo screening of drugs useful in the treatment of human or animal pathology linked to a dysfunction of the expression of the MCM8 gene, said method comprising contacting of the potential drugs with cells selected from the group comprising cancer cells, cells wherein recombinant and/or mutated active forms of MCM8 helicase are introduced, and transformed cells selected from the group comprising liver, brain, muscle, skin and gut cells wherein an increase of the expression of an active form of MCM8 helicase is induced by transformation of said cells with recombinant and/or mutated forms of the human or murine or xenopus MCM8 gene, or of parts of said gene, or of transcripts thereof, and screening the drugs able to inhibit the proliferation of said cells.

43. A method for the in vitro or ex vivo screening of drugs useful in the treatment of human or animal pathology linked to a dysfunction of the expression of the MCM8 gene, said method comprising contacting of the potential drugs with transformed cells selected from the group comprising liver, brain, muscle, skin and gut cells wherein an increase of the expression of an inactive MCM8 helicase is induced by transformation of said cells with recombinant and/or mutated forms of the human or murine or xenopus MCM8 gene, or of parts of said gene, or of transcripts thereof, or wherein a decrease of the expression of the MCM8 helicase is induced by transformation of said cells with antisense nucleic acids able to hybridize with part of said gene or transcripts, or of silencing RNA derived from parts of said transcripts and able to repress said MCM8 gene, and screening the drugs able to stimulate the proliferation of said transformed cells.

44. A method for the in vitro or ex vivo production of catalytically. active MCM8 helicase in foreign expression systems, selected from the group comprising insect cells (Sf9) or equivalent and in vitro systems for coupled transcription/translation of the MCM8 cDNA, selected from the group comprising rabbit reticulocytes systems, lysate of E. coli cells, translation of the MCM8 mRNA into xenopus oocyte and egg extracts, under form of a tagged recombinant protein, comprising the steps of:
lysis of cells expressing MCM8 proteins in the following buffer or equivalent, 20 mM TrisHCl pH 8.5, 100 mM KCl, 5 mM □-mercaptoethanol, 5-10 mM imidazole, 10% glycerol (v/v) proteases inhibitors;
purification of the soluble MCM8 proteins by nickel affinity chromatography technology or equivalent or similar affinity chromatography technology;
elution of bound proteins in 10 mM TrisHCl pH 8.5; 100 mM KCl; 5 mM □-mercaptoethanol; 100-250 mM imidazole, 10% glycerol (v/v) proteases inhibitors;
supplementation of purified MCM8 proteins, with or without cleaved tag, with 0.1 mg/ml of BSA;
desaltation on a Bio-spin P30 column (Biorad) equilibrated with 20 mM TrisHCl pH 7.4, 150 mM NaCl, 0.5 mM EDTA, 1 mM DTT, 0.01% Triton X-100 for helicase and ATPase activities, or in XB (100 mM KCl, 0.1 mM CaCl₂, 2 mM MgCl₂, 10 mM Hepes-KOH, 50 mM sucrose, pH 7.7) for egg extracts reconstitution experiments; and
supplementation of the protein with 25% glycerol and storage at −20° C.

45. A DNA vector containing an MCM8 gene selected from the group comprising genes of SEQ ID NO: 1 or SEQ ID NO: 3 or SEQ ID NO: 5 or SEQ ID NO: 7 or SEQ ID NO: 9 or SEQ ID NO: 11 or SEQ ID NO: 13 or SEQ ID NO: 15 or SEQ ID NO: 17 or SEQ ID NO: 19 or SEQ ID NO: 21, and a mutated form of the MCM8 gene according to claim 35, operatively linked to regulatory sequences.

46. A host cell transformed with a DNA vector according to claim 45.

47. A recombinant protein obtained by the expression of the DNA vector according to claim 45.

48. An antibody or antigen-binding fragment which binds to an MCM8 protein or part of an MCM8 protein or to a modified active MCM8 protein or to a modified part of an MCM8 protein, selected from the group comprising polypeptides comprising the totality or part of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ ID NO: 22.

49. A monoclonal and polyclonal antibodies directed against an MCM8 helicase or against polypeptides comprising part of an MCM8 helicase selected from the group comprising polypeptides comprising the totality or part of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ ID NO: 22.

50. A pharmaceutical preparations comprising an MCM8 helicase or a polypeptide comprising part of an MCM8 helicase selected from the group comprising polypeptides comprising the totality or part of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ ID NO: 22 or a mutated form of the MCM8 helicase according to claim 35.

51. Humanized immunoglobulin chains having specificity for an MCM8 helicase selected from the group comprising polypeptides of SEQ ID NO: 2 or SEQ ID NO: 4 or SEQ ID NO: 6 or SEQ ID NO: 8 or SEQ ID NO: 10 or SEQ ID NO: 12 or SEQ ID NO: 14 or SEQ ID NO: 16 or SEQ ID NO: 18 or SEQ ID NO: 20 or SEQ ID NO: 22.

52. A method for inhibiting cell proliferation or allowing a better replication of the DNA, comprising administering an agonist or antagonist of an MCM8 helicase in a way that the agonist or antagonist enters the cell, said antagonist causing the inhibition of DNA replication and said agonist contributing to the restoration of cell replication or to the ability of the cell to replicate DNA in unfavorable conditions.

53. A method for inhibiting cell proliferation or allowing a better replication of the DNA in vitro or ex vivo, comprising administering an agonist or antagonist of an MCM8 helicase in a way that the agonist or antagonist enters the cell, said antagonist causing the inhibition of DNA replication and said agonist contributing to the restoration of cell replication or to the ability of the cell to replicate DNA in unfavorable conditions.


SEQUENCE LISTING (42 sequences)

SEQ ID NO: 1 - DNA, 2505 bp, Xenopus sp.
  CDS (1)..(2505): First CDS region
atgagtcagggttggagaggaggatctggaggatggagaggaggaggaggaggaggaggcaatccatatgcaggagcttggagaggtcggccatggagaggaaggggtcagggtggaacctggagcagaaacaatggaagagatcctgtttgttttgctccccctaagccgcagctaacacagactacactagataaatacatcccctacaaaggctggaagctttatttttctgaagcttacagtgacaactccccatttttggagaaggttcgtgcctttgagaagtttttcaaaaaacaaatcgaactttatgacaaggatgaaattgaaagaaaaggaagtattcttgtggactacaaagaactcctgcaagatgaagacctttctgcgtccatcccattgtcctcagagcttaaagacatgcctgaaaaagtactggagtgtatgggtctagctatacatcaggtattaacaaaagatcttgagacacatgcagcagatctgcagcagcaggagggcctcagaacagaggaagctcctatagttaatgtgcccttcattcacgccagggtatttaattacgatacactgacatctctaaaaaatctcagggcaagtttgtacggcaaatatgttgctttgcgtgggacggtggttcgcgtcggtaacatcaagccgttgtgcactaaaatggctttttcctgcaacatgtgcggcgacattcagtgttttcctcttcccgatggaaagtacactgtgccaacaaagtgccctgtgccagaatgtcggggtcgctcatttactgcaaacagaagttccccgctcacagtaactgtggactggcaaaccatcaaagtccaggagctaatgtcggatgatcagcgagaggcaggtcggattccaagaaccgttgagtgtgaattaattcaggatctggtagacagctgtgttcctggcgacatgattactgtaacaggcattgtcaaagtatcaaatacaagagacggtggctttaagaataaaaataacaaatgcatgttccttctgtacatcgaggccaattctgtcagtaacagcaaaggacaaaaaggcaaaagtacagaagatagtggcaatcatggagcctctatggatttttctctcaaagacctctatgctatccaggaaattcagtcccaagaaaatctctttcagttaatagtcaactcactttgtcctacaatatatggacatgagttggtcaaagccggactgtcactggcactttttgggggctgtcagaagtacgcagatgataaaaacaggattcccatccgaggagatcctcatatacttgtggtaggagaccctggtttagggaaaagtcagatgttgcaagcagtgtgcaacgtggctcctcgtggtgtttatgtttgtggaaacactaccaccacctccggactgaccgtcaccttatccagagacactacaactggagactttggcttggaagccggggcactcgtcctcggagatcaaggtatctgtggaatagatgagtttgataagatggggaaccagcaccaggcattactggaagcgatggaacagcagagcattagtttggctaaagctggaattgtgtgcagcctcccggccaggacatccatcatcgctgctgctaaccctgttggggggcactataacaaaggaaaaaccgtgtctgagaatttaaaaatgggaagtgctttgctttctcgttttgacttagttttcattcttgtcgataccccaaatgaagaccatgatcacctgctgtcagagcatgtaatggcgatgagatctggggctaaggaaatacaaagtgtggatataacacgtataaatacccagaactccaacacctccattctagaggtgccctctgaaaggcccttaggggaaagactgaagctgaggactggggaacactttgatgctctacctcaccagctattaaggaaatttgttggctacgctcgccagtatgtccacccaagtctgtctcctgatgcagctcagattcttcaggacttctatcttgaattaagaaaacaaaaccaaggcatagacagtactcccattaccacaagacaactggagtctctcatcagactgacagaggcacgagcaagactggagttaagagaaaaggccactaaagaagacgctgaagaggtggttcaaataatgaagtacagcctgctgggcaccttctctgatgagtttggaaagctggactttcagcgctcacaacacggttcaggaatgagcaatcgatcaaaagcaaaaaagtttgtatccgccctcaatagagtcgccgaacaaacctacaacaatctgtttgagtttcagcaacttcgccaaatcgccagagaattacaaatacaggttattgactttgaagcttttataggctccttaaatgaccaagggtatctcctgaagaagggcccccgagtcttccagctccagaccatgtga

SEQ ID NO: 2 - protein, 834 aa, Xenopus sp.
MSQGWRGGSGGWRGGGGGGGNPYAGAWRGRPWRGRGQGGTWSRNNGRDPVCFAPPKPQLTQTTLDKYIPYKGWKLYFSEAYSDNSPFLEKVRAFEKFFKKQIELYDKDEIERKGSILVDYKELLQDEDLSASIPLSSELKDMPEKVLECMGLAIHQVLTKDLETHAADLQQQEGLRTEEAPIVNVPFIHARVFNYDTLTSLKNLRASLYGKYVALRGTVVRVGNIKPLCTKMAFSCNMCGDIQCFPLPDGKYTVPTKCPVPECRGRSFTANRSSPLTVTVDWQTIKVQELMSDDQREAGRIPRTVECELIQDLVDSCVPGDMITVTGIVKVSNTRDGGFKNKNNKCMFLLYIEANSVSNSKGQKGKSTEDSGNHGASMDFSLKDLYAIQEIQSQENLFQLIVNSLCPTIYGHELVKAGLSLALFGGCQKYADDKNRIPIRGDPHILVVGDPGLGKSQMLQAVCNVAPRGVYVCGNTTTTSGLTVTLSRDTTTGDFGLEAGALVLGDQGICGIDEFDKMGNQHQALLEAMEQQSISLAKAGIVCSLPARTSIIAAANPVGGHYNKGKTVSENLKMGSALLSRFDLVFILVDTPNEDHDHLLSEHVMAMRSGAKEIQSVDITRINTQNSNTSILEVPSERPLGERLKLRTGEHFDALPHQLLRKFVGYARQYVHPSLSPDAAQILQDFYLELRKQNQGIDSTPITTRQLESLIRLTEARARLELREKATKEDAEEVVQIMKYSLLGTFSDEFGKLDFQRSQHGSGMSNRSKAKKFVSALNRVAEQTYNNLFEFQQLRQIARELQIQVIDFEAFIGSLNDQGYLLKKGPRVFQLQTM

SEQ ID NO: 3 - DNA, 2841 bp, Homo sapiens
  CDS (1)..(2472): First CDS region
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtcagagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcaaatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacttcaccaagttagggcctcctgggtttattgcagattaaagccatctcagtgaagatatgcgtgcacgcacagacagacagacacacacacacacacacacacacacacacacacacacacacacacagtcaaatactgttctctgaaaaatgatgtcccaaaagtattataataggaaaaaagcattaaatataataaactaatttaagaagtgataaagtctccagatgcagtagctcacactgtaatcacagtgactcaggaggctgaggtgagaggattccttgaggccagggttcgagaccaaccttgggcaacatagcaagaccccatttcttaaaaaaaaaaaaaaaaaaaaa

SEQ ID NO: 4 - protein, 824 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTM

SEQ ID NO: 5 - DNA, 3279 bp, Homo sapiens
  CDS (1)..(2472): First CDS region
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtcagagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcaaatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacttcaccaagttagggcctcctgggtttattgcagattaaagccatctcagtgaagatatgcgtgcacgcacagacagacagacacacacacacacacacacacacacacacacacacacacacacagtcaaatactgttctctgaaaaatgatgtcccaaaagtattataataggaaaaaagcattaaatataataaactaatttaagaagtgataaagtctccagatgcagtagctcacactgtaatcacagtgactcaggaggctgaggtgagaggattccttgaggccagggttcgagaccaaccttgggcaacatagcaagaccccatttcttaaaaaaaaaaaaaaaaaatttaaacttagctgggtatggtggcacatgcctatagtctcagctacttgtgaggctgaggcaggaggattctttgagcccaggagtttgaggttacagtgagccacaatcacaccaatcactgcactccagcctgggcaataaagtaactcttgactcaaaaaaataaaaaaaattgtagtggtagccatgtgttaattgttaaataaattctccaaagggctaaaagtaaattacttataaattttttatagttgtatttttgacctgccttttatatgtatgaatatttcatagttttgcatatcagatgtaggcatacagacaaatacataaaccaatgaatatattacatattctgtgttccaataaaactttatttatggacactaaaatttgaatttcataaaattttcccatgtcaagaatacaaaaaaaaaaaa

SEQ ID NO: 6 - protein, 824 aa, Homo sapiens; identical to SEQ ID NO: 4

SEQ ID NO: 7 - DNA, 3327 bp, Homo sapiens
  CDS (1)..(2523): First CDS region
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtcagagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcaaatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacttcaccaagttagggcctcctgggtttattgcagattaaagccatctcagtgaagatatgcgtgcacgcacagacagacagacacacacacacacacacacacacacacacacacacacacacacagtcaaatactgttctctgaaaaatgatgtcccaaaagtattataataggaaaaaagcattaaatataataaactaatttaagaagtgataaagtctccagatgcagtagctcacactgtaatcacagtgactcaggaggctgaggtgagaggattccttgaggccagggttcgagaccaaccttgggcaacatagcaagaccccatttcttaaaaaaaaaaaaaaaaaatttaaacttagctgggtatggtggcacatgcctatagtctcagctacttgtgaggctgaggcaggaggattctttgagcccaggagtttgaggttacagtgagccacaatcacaccaatcactgcactccagcctgggcaataaagtaactcttgactcaaaaaaataaaaaaaattgtagtggtagccatgtgttaattgttaaataaattctccaaagggctaaaagtaaattacttataaattttttatagttgtatttttgacctgccttttatatgtatgaatatttcatagttttgcatatcagatgtaggcatacagacaaatacataaaccaatgaatatattacatattctgtgttccaataaaactttatttatggacactaaaatttgaatttcataaaattttcccatgtcaagaatacaaaaaaaaaaaa

SEQ ID NO: 8 - protein, 840 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTM

SEQ ID NO: 9 - DNA, 2888 bp, Homo sapiens
  CDS (1)..(2520): First CDS region
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtcagagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcttaggaaaaagtcaaatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacttcaccaagttagggcctcctgggtttattgcagattaaagccatctcagtgaagatatgcgtgcacgcacagacagacagacacacacacacacacacacacacacacacacacacacacacacacagtcaaatactgttctctgaaaaatgatgtcccaaaagtattataataggaaaaaagcattaaatataataaactaatttaagaagtgataaagtctccagatgcagtagctcacactgtaatcacagtgactcaggaggctgaggtgagaggattccttgaggccagggttcgagaccaaccttgggcaacatagcaagaccccatttcttaaaaaaaaaaaaaaaaaaaa

SEQ ID NO: 10 - protein, 840 aa, Homo sapiens; identical to SEQ ID NO: 8

SEQ ID NO: 11 - DNA, 2574 bp, Homo sapiens
  CDS (1)..(2208): First CDS region
catattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtcagagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcaaatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacttcaccaagttagggcctcctgggtttattgcagattaaagccatctcagtgaagatatgcgtgcacgcacagacagacagacacacacacacacacacacacacacacacacacacacacacagtcaaatactgttctctgaaaaatgatgtcccaaaagtattataataggaaaaaagcattaaatataataaactaatttaagaagtgataaagtctccagatgcagtagctcacactgtaatcacagtgactcaggaggctgaggtgagaggattccttgaggccagggttcgagaccaaccttgggcaacatagcaagaccccatttcttaaaaaaaaaaaaaaaaaaaaaaaaa

SEQ ID NO: 12 - protein, 735 aa, Homo sapiens
HIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTM

SEQ ID NO: 13 - DNA, 2844 bp, Homo sapiens
  CDS (1)..(2472): First CDS region
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtcagagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcaaatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacttcaccaagttagggcctcctgggtttattgcagattaaagccatctcagtgaagatatgcgtgcacgcacagacagacagacacacacacacacacacacacacacacacacacacacacacacacagtcaaatactgttctctgaaaaatgatgtcccaaaagtattataataggaaaaaagcattaaatataataaactaatttaagaaggtgataaagtctccagatgcaggtaggctcacactgtaatcacagtgactcaggaggctgaggtgagaggattccttgaggccagggttcgagaccaaccttgggcaacatagcaagaccccatttcttaaaaaaaaaaaaaaaaaaaaa

SEQ ID NO: 14 - protein, 824 aa, Homo sapiens; identical to SEQ ID NO: 4

SEQ ID NO: 15 - DNA, 2877 bp, Homo sapiens
  CDS (1)..(2520): First CDS region
atgaatggagagtatagaggcagaggatttggacgaggaagatttcaaagctggaaaaggggaagaggtggtgggaacttctcaggaaaatggagagaaagagaacacagacctgatctgagtaaaaccacaggaaaacgtacttctgaacaaaccccacagtttttgctttcaacaaagaccccacagtcaatgcagtcaacattggatcgattcataccatataaaggctggaagctttatttctctgaagtttacagcgatagctctcctttgattgagaagattcaagcatttgaaaaatttttcacaaggcatattgatttgtatgacaaggatgaaatagaaagaaagggaagtattttggtagattttaaagaactgacagaaggtggtgaagtaactaacttgataccagatatagcaactgaactaagagatgcacctgagaaaaccttggcttgcatgggtttggcaatacatcaggtgttaactaaggaccttgaaaggcatgcagctgagttacaagcccaggaaggattgtctaatgatggagaaacaatggtaaatgtgccacatattcatgcaagggtgtacaactatgagcctttgacacagctcaagaatgtccgagcaaattactatggaaaatacattgctctaagagggacagtggttcgtgtcagtaatataaagcctctttgcaccaagatggcttttctttgtgctgcatgtggagaaattcagagctttcctcttccagatggaaaatacagtcttcccacaaagtgtcctgtgcctgtgtgtcgaggcaggtcatttactgctctccgcagctctcctctcacagttacgatggactggcagtcaatcaaaatccaggaattgatgtctgatgatcagagagaagcaggtcggattccacgaacaatagaatgtgagcttgttcatgatcttgtggatagctgtgtcccgggagacacagtgactattactggaattgtcaaagtctcaaatgcggaagaaggttctcgaaataagaatgacaagtgtatgttccttttgtatattgaagcaaattctattagtaatagcaaaggacagaaaacaaagagttctgaggatgggtgtaagcatggaatgttgatggagttctcacttaaagacctttatgccatccaagagattcaagctgaagaaaacctgtttaaactcattgtcaactcgctttgccctgtcatttttggtcatgaacttgttaaagcaggtttggcattagcactctttggaggaagccagaaatacgcagatgacaaaaacagaattccaattcggggagacccccacatccttgttgttggagatccaggcctaggaaaaagtcagatgctacaggcagcgtgcaatgttgccccacgtggcgtgtatgtttgtggtaacaccacgaccacctctggtctgacggtaactctttcaaaagatagttcctctggagattttgctttggaagctggtgccctggtacttggtgatcaaggtatttgtggaatcgatgaatttgataagatggggaatcaacatcaagccttgttggaagccatggagcagcaaagtattagtcttgctaaggctggtgtggtttgtagccttcctgcaagaacttccattattgctgctgcaaatccagttggaggacattacaataaagccaaaacagtttctgagaatttaaaaatggggagtgcactactatccagatttgatttggtctttatcctgttagatactccaaatgagcatcatgatcacttactctctgaacatgtgattgcaataagagctggaaagcagagaaccattagcagtgccacagtagctcgtatgaatagtcaagattcaaatacttccgtacttgaagtagtttctgagaagccattatcagaaagactaaaggtggttcctggagaaacaatagatcccattccccaccagctattgagaaagtacattggctatgctcggcagtatgtgtacccaaggctatccacagaagctgctcgagttcttcaagatttttaccttgagctccggaaacagagccagaggttaaatagctcaccaatcactaccaggcagctggaatctttgattcgtctgacagaggcacgagcaaggttggaattgagagaggaagcaaccaaagaagacgctgaggatatagtggaaattatgaaatatagcatgctaggaacttactctgatgaatttgggaacctagattttgagcgatcccagcatggttctggaatgagcaacaggtcaacagcgaaaagatttatttctgctctcaacaacgttgctgaaagaacttataataatatatttcaatttcatcaacttcggcagattgccaaagaactaaacattcaggttgctgattttgaaaattttattggatcactaaatgaccagggttacctcttgaaaaaaggcccaaaagtttaccagcttcaaactatgtaaaaggacnttcaccaagttagggcctcctgggtttattgcagattaaagccatctcagtgaagatatgcgtgcacgcacagacagacagacacacacacacacacacacacacacacacacacacacacacagtcaaatactgttctctgaaaaatgatgtcccaaaagtattataataggaaaaaagcattaaatataataaactaatttaagaagtgataaagtctccagatgcagtagctcacactgtaatcacagtgactcaggaggctgaggtgagaggattccttgaggccagggttcgagaccaaccttgggcaacatagcaagaccccatttcttaaaaaaaaaaaa

SEQ ID NO: 16 - protein, 840 aa, Homo sapiens; identical to SEQ ID NO: 8

SEQ ID NO: 17 - DNA, 2612 bp, Mus musculus
  CDS (1)..(2499): First CDS region
atgagtggtgcttatagaggcagaggttttggacgaggaagattccaaagctggaaaagaggaagaggtggtgggaacttctcaggaaggtggagagaaagagaaaacagagttgacctgaatgaagcttcaggaaagcacgcttctgcacaagcctcacaaccgttacttcaacagtcaacattggatcagtttatcccgtataagggctggaagctttacttctctgaagtttacagtaataactctccctttattgagaagattcaagcatttgagaaatttttcacaaggcatatcgacttatacgataaggatgaaatagaaagaaaaggaagcattttggtggattttaaagaactgacaaaagctgatgaaataactaacttgatacccgatatagaaaacgcactaagagatgcccctgagaaaacactggcgtgtatggggctggcaatacatcaggtattaactaaggaccttgaaaggcatgccgccgaattacaagctcaagagggattgtctaacggtggagagacaatggtaaatgtaccacatatttatgcaagagtgtacaactatgagcccttgacacacctcaagaatatccgagcaacttgctatgggaaatacatctctatacgagggactgtggtccgggtcagcaacataaagcctctttgcaccaacatggcttttcagtgcgctgcatgtggagagattcagagctttcctctgccagatggaaaatacacccttcctacaaagtgtcctgtgcctgcgtgccgagggaggtcatttgctccactgcgcagctctcctctcacagttacactggactggcagttgatcaaaatccaggagctgatgtctgatgcacagagagaagctggtcggatccctcggacgatagaatgtgaacttgttcacgatcttgtagatagttgtgtcccaggagatacagtgactgttactggaattgtcaaagtctccaattctgaagaaggttctcgaaataagaatgataagtgcatgttccttttgtacattgaggcaaattctgttagcaacagcaaggggccgaaagcacagactgcagaagatggttgcaagcatgggacactgatggagttctcccttaaagacctctatgccatccgagagatccaggctgaagagaacctgctcaagctcgttgtcaactcactctgtcctgtcatttttggtcatgaactcgtcaaagcaggtctgacgttagcactctttggcggtagccagaagtacgcagatgacaaaaacagaattcccattcgaggagacccacatgtcctgattgttggagatccaggcttggggaagagtcagatgctacaggcagcatgcaacgtggcgccacgtggtgtgtatgtttgtggaaataccaccaccagctctggtctcactgtgactctttcaaaggacagttcctctggagattttgctttggaagctggtgcccttgtacttggtgaccaaggcatctgtggaatagatgaatttgataaaatggggaaccaacatcaagccctgttagaagccatggaacagcagagtattagccttgcgaaggctggggtagtttgcagcctccctgcaagaacttccattgttgctgctgcaaatccagtcggaggacactacaataaagccagaacggtttctgagaatttaaagatggggagtgccctactgtccagattcgatttggtctttatcttgttagatacgccaaatgaacagcatgaccacttactttctgaacatgtgatcgcgataagagctgggaagcagaaagcggttagcagtgccacagtcactcgtgtgctcagtcaagactcaaatacttctgtacttgaagtggtttctgagaaaccattatcagaaagactaaaggtggctcctggagaacagacagatccgattccacatcagctgttgaggaagtacattggctatgcacggcagtatgtccacccaaggttatctacggacgctgctcaggcccttcaggatttctacctggagctccgcaagcagagccagcgcgtgggcagctcacccatcaccactcggcagctggagtctttgatccgtctgacagaggcacgggcaaggttagaattgagagaggaagcaactagagaagatgctgaagatataattgaaattatgaagcatagcatgctaggaacttactcagatgaattcggaaacctggactttgagcgatcccagcatggctctgggatgagcaacaggtcaacagcaaaaagatttatttctgctctcaacagcattgctgaaagaacttataacaacatatttcaatatcatcaacttcgtcagattgctaaagaactaaacattcaggttgccgattttgaaaacttcattggatcactgaatgaccaaggttatctcttgaagaaaggcccaaagatttaccaacttcaaactatgtgaaagactttaccacatcagggcttcctgtggttcaaaatagttcagaatcatcgtaattacaaaactgaaaataaaagttctgaaaaatgaaaaaaaaaaaaaaaaaaaaa

SEQ ID NO: 18 - protein, 833 aa, Mus musculus
MSGAYRGRGFGRGRFQSWKRGRGGGNFSGRWRERENRVDLNEASGKHASAQASQPLLQQSTLDQFIPYKGWKLYFSEVYSNNSPFIEKIQAFEKFFTRHIDLYDKDEIERKGSILVDFKELTKADEITNLIPDIENALRDAPEKTLACMGLAIHQVLTKDLERHAAELQAQEGLSNGGETMVNVPHIYARVYNYEPLTHLKNIRATCYGKYISIRGTVVRVSNIKPLCTNMAFQCAACGEIQSFPLPDGKYTLPTKCPVPACRGRSFAPLRSSPLTVTLDWQLIKIQELMSDAQREAGRIPRTIECELVHDLVDSCVPGDTVTVTGIVKVSNSEEGSRNKNDKCMFLLYIEANSVSNSKGPKAQTAEDGCKHGTLMEFSLKDLYAIREIQAEENLLKLVVNSLCPVIFGHELVKAGLTLALFGGSQKYADDKNRIPIRGDPHVLIVGDPGLGKSQMLQAACNVAPRGVYVCGNTTTSSGLTVTLSKDSSSGDFALEAGALVLGDQGICGIDEFDKMGNQHQALLEAMEQQSISLAKAGVVCSLPARTSIVAAANPVGGHYNKARTVSENLKMGSALLSRFDLVFILLDTPNEQHDHLLSEHVIAIRAGKQKAVSSATVTRVLSQDSNTSVLEVVSEKPLSERLKVAPGEQTDPIPHQLLRKYIGYARQYVHPRLSTDAAQALQDFYLELRKQSQRVGSSPITTRQLESLIRLTEARARLELREEATREDAEDIIEIMKHSMLGTYSDEFGNLDFERSQHGSGMSNRSTAKRFISALNSIAERTYNNIFQYHQLRQIAKELNIQVADFENFIGSLNDQGYLLKKGPKIYQLQTM

SEQ ID NO: 19 - DNA, 3058 bp, Mus musculus
  CDS (1)..(2415): First CDS region
atgagtggtgcttatagaggcagaggttttggacgaggaagattccaaagctggaaaagaggaagaggtggtgggaacttctcaggaaggtggagagaaagagaaaacagagttgacctgaatgaagcttcaggaaagcacgcttctgtttacagtaataactctccctttattgagaagattcaagcatttgagaaatttttcacaaggcatatcgacttatacgataaggatgaaatagaaagaaaaggaagcattttggtggattttaaagaactgacaaaagctgatgaaataactaacttgatacccgatatagaaaacgcactaagagatgcccctgagaaaacactggcgtgtatggggctggcaatacatcaggtattaactaaggaccttgaaaggcatgccgccgaattacaagctcaagagggattgtctaacggtggagagacaatggtaaatgtaccacatatttatgcaagagtgtacaactatgagcccttgacacacctcaagaatatccgagcaacttgctatgggaaatacatctctatacgagggactgtggtccgggtcagcaacataaagcctctttgtaccaacatggcttttcagtgcgctgcatgtggagagattcagagctttcctctgccagatggaaaatacacccttcctacaaagtgtcctgtgcctgcgtgccgagggaggtcatttgctccactgcgcagctctcctctcacagttacactggactggcagttgatcaaaatccaggagctgatgtctgatgcacagagagaagctggtcggatccctcggacgatagaatgtgaacttgttcacgatcttgtagatagttgtgtcccaggagatacagtgactgttactggaattgtcaaagtctccaattctgaagaaggttctcgaaataagaatgataagtgcatgttccttttgtacattgaggcaaattctgttagcaacagcaaggggccgaaagcacagactgcagaagatggttgcaagcatgggacactgatggagttctcccttaaagacctctatgccatccgagagatccaggctgaagagaacctgctcaagctcgttgtcaactcactctgtcctgtcatttttggtcatgaactcgtcaaagcaggtctgacgttagcactctttggcggtagccagaagtacgcagatgacaaaaacagaattcccattcgaggagacccacatgtcctgattgttggagatccaggcttggggaagagtcagatgctacaggcagcatgcaacgtggcgccacgtggtgtgtatgtttgtggaaataccaccaccagctctggtctcactgtgactctttcaaaggacagttcctctggagattttgctttggaagctggtgcccttgtacttggtgaccaaggcatctgtggaatagatgaatttgataaaatggggaaccaacatcaagccctgttagaagccatggaacagcagagtattagccttgcgaaggctggggtagtttgcagcctccctgcaagaacttccattattgctgctgcaaatccagtcggaggacactacaataaagccagaacggtttctgagaatttaaagatggggagtgccctactgtccagattcgatttggtctttatcttgttagatacgccaaatgaacagcatgaccacttactttctgaacatgtgatcgcgataagagctgggaagcagaaagcggttagcagtgccacagtcactcgtgtgctcagtcaagactcaaatacttctgtacttgaagtggtttctgagaaaccattatcagaaagactaaaggtggctcctggagaacagacagatccgattccacatcagctgttgaggaagtacattggctatgcacggcagtatgtccacccaaggttatctacggacgctgctcaggcccttcaggatttctacctggagctccgcaagcagagccagcgcgtgggcagctcacccatcaccactcggcagctggagtctttgatccgtctgacagaggcacgggcaaggttagaattgagagaggaagcaactagagaagatgctgaagatataattgaaattatgaagcatagcatgctaggaacttactcagatgaattcggaaacctggactttgagcgatcccagcatggctctgggatgagcaacaggtcaacagcaaaaagatttatttctgctctcaacagcattgctgaaagaacttataacaacatatttcaatatcatcaacttcgtcagattgctaaagaactaaacattcaggttgccgattttgaaaacttcattggatcactgaatgaccaaggttatctcttgaagaaaggcccaaagatttaccaacttcaaactatgtgaaagactttaccacatcagggcttcctgtggttcaaaatagttcagaatcatcgtaattacaaaactgaaaataaaagttctgaaaaatgacacaactcctataacagaataaaggcctatccataatttatccagaagagtaagaagagtaaattgtttccattattttatctaaatagcataagcatatgcttttaactcatcatatttttagtagtgctttttagttttgtatatcagatgtctagcaggcattcagctaatgcaaaaacagattagtatgtgttcaggtgaacttttacttaaaacattaaactttggctctcctaaattgttcaggtcacaaaaactcttcattcatttttaactttatgattatgggcagtgggtctgacttggcttctcatttgaaagtagtggaaatttattgacttaattagaagtcttttctggggctagagagatgactcagtggttaagagcactgactgctcttccataggtcctgagttcaagtcccagcaaccacatggtgactcacaaccatctgtaatgggatccaatgccctcttctcgtgtctctgaagagagtaacagtgcactcacataaataaatcttaaaaaaaaaaaaaaaaaaaaa

SEQ ID NO: 20 - protein, 805 aa, Mus musculus
MSGAYRGRGFGRGRFQSWKRGRGGGNFSGRWRERENRVDLNEASGKHASVYSNNSPFIEKIQAFEKFFTRHIDLYDKDEIERKGSILVDFKELTKADEITNLIPDIENALRDAPEKTLACMGLAIHQVLTKDLERHAAELQAQEGLSNGGETMVNVPHIYARVYNYEPLTHLKNIRATCYGKYISIRGTVVRVSNIKPLCTNMAFQCAACGEIQSFPLPDGKYTLPTKCPVPACRGRSFAPLRSSPLTVTLDWQLIKIQELMSDAQREAGRIPRTIECELVHDLVDSCVPGDTVTVTGIVKVSNSEEGSRNKNDKCMFLLYIEANSVSNSKGPKAQTAEDGCKHGTLMEFSLKDLYAIREIQAEENLLKLVVNSLCPVIFGHELVKAGLTLALFGGSQKYADDKNRIPIRGDPHVLIVGDPGLGKSQMLQAACNVAPRGVYVCGNTTTSSGLTVTLSKDSSSGDFALEAGALVLGDQGICGIDEFDKMGNQHQALLEAMEQQSISLAKAGVVCSLPARTSIIAAANPVGGHYNKARTVSENLKMGSALLSRFDLVFILLDTPNEQHDHLLSEHVIAIRAGKQKAVSSATVTRVLSQDSNTSVLEVVSEKPLSERLKVAPGEQTDPIPHQLLRKYIGYARQYVHPRLSTDAAQALQDFYLELRKQSQRVGSSPITTRQLESLIRLTEARARLELREEATREDAEDIIEIMKHSMLGTYSDEFGNLDFERSQHGSGMSNRSTAKRFISALNSIAERTYNNIFQYHQLRQIAKELNIQVADFENFIGSLNDQGYLLKKGPKIYQLQTM

SEQ ID NO: 21 - DNA, 3058 bp, Mus musculus; identical to SEQ ID NO: 19
  CDS (1)..(2415): First CDS region

SEQ ID NO: 22 - protein, 805 aa, Mus musculus; identical to SEQ ID NO: 20

SEQ ID NO: 23 - DNA, 24 bp, Xenopus sp.
  CDS (1)..(24): First CDS region
ggagaccctggtttagggaaaagt

SEQ ID NO: 24 - protein, 8 aa, Xenopus sp.
GDPGLGKS

SEQ ID NO: 25 - DNA, 12 bp, Xenopus sp.
  CDS (1)..(12): First CDS region
gatgagtttgat

SEQ ID NO: 26 - protein, 4 aa, Xenopus sp.
DEFD

SEQ ID NO: 27 - DNA, 24 bp, Homo sapiens
  CDS (1)..(24): First CDS region
ggagatccaggcttaggaaaaagt

SEQ ID NO: 28 - protein, 8 aa, Homo sapiens
GDPGLGKS

SEQ ID NO: 29 - DNA, 12 bp, Homo sapiens
  CDS (1)..(12): First CDS region
gatgaatttgat

SEQ ID NO: 30 - protein, 4 aa, Homo sapiens
DEFD

SEQ ID NO: 31 - DNA, 27 bp, Homo sapiens
  CDS (1)..(27): First CDS region
ggagatccaggcctaggaaaaagtcaa

SEQ ID NO: 32 - protein, 9 aa, Homo sapiens
GDPGLGKSQ

SEQ ID NO: 33 - DNA, 12 bp, Homo sapiens
  CDS (1)..(12): First CDS region
gatgaatttgat

SEQ ID NO: 34 - protein, 4 aa, Homo sapiens
DEFD

SEQ ID NO: 35 - DNA, 24 bp, Mus musculus
  CDS (1)..(24): First CDS region
ggagatccaggcttggggaagagt

SEQ ID NO: 36 - protein, 8 aa, Mus musculus
GDPGLGKS

SEQ ID NO: 37 - DNA, 12 bp, Mus musculus
  CDS (1)..(12): First CDS region
gatgaatttgat

SEQ ID NO: 38 - protein, 4 aa, Mus musculus
DEFD

SEQ ID NO: 39 - DNA, 24 bp, Mus musculus
  CDS (1)..(24): First CDS region
ggagatccaggcttggggaagagt

SEQ ID NO: 40 - protein, 8 aa, Mus musculus
GDPGLGKS

SEQ ID NO: 41 - DNA, 12 bp, Mus musculus
  CDS (1)..(12): First CDS region
gatgaatttgat

SEQ ID NO: 42 - protein, 4 aa, Mus musculus
DEFD